(19) United States
(12) Patent Application Publication
(10) Pub. No.: US 2015/0141830 A1
(43) Pub. Date: May 21, 2015

(54) ULTRASONIC DIAGNOSTIC APPARATUS AND CONTROL METHOD

(71) Applicants: Kabushiki Kaisha Toshiba, Minato-ku (JP); Toshiba Medical Systems Corporation, Otawara-shi (JP)

(72) Inventors: Akihiro KAKEE, Nasushiobara (JP); Takeshi FUKASAWA, Nasushiobara (JP)

(73) Assignees: Kabushiki Kaisha Toshiba, Minato-ku (JP); Toshiba Medical Systems Corporation, Otawara-shi (JP)

(21) Appl. No.: 14/537,142

(22) Filed: Nov. 10, 2014

(30) Foreign Application Priority Data
Nov. 19, 2013 (JP) 2013-239381

(51) Int. Cl. A61B 8/14; A61B 8/00; A61B 8/08

(52) U.S. Cl. CPC A61B 8/145; A61B 8/5207; A61B 8/54; A61B 8/5253
USPC 600/447

(57) ABSTRACT

An ultrasonic diagnostic apparatus according to an embodiment includes a transmitter/receiver, a signal processor, an image generator, and a controller. The transmitter/receiver executes ultrasonic transmission/reception sets a plurality of times on an identical scanning line by changing transmission conditions, and generates a plurality of sets of reception signals, the ultrasonic transmission/reception sets including a plurality of ultrasonic transmissions/receptions on the identical scanning line serving as a unit. The signal processor combines the reception signals in each of the plurality of the sets, and generates a plurality of composite signals corresponding to each of the plurality of the sets. The image generator generates ultrasonic image data using the composite signals. The controller controls an order of ultrasonic transmissions/receptions executed by the transmitter/receiver such that previous transmissions of respective transmissions corresponding to the reception signals in one set combined by the signal processor have an identical transmission condition but different phase polarities.

### CROSS-REFERENCE TO RELATED APPLICATIONS

This application is based upon and claims the benefit of priority from Japanese Patent Application No. 2013-239381, filed on Nov. 19, 2013, the entire contents of all of which are incorporated herein by reference.

### FIELD

Embodiments described herein relate generally to an ultrasonic diagnostic apparatus and a control method.

### BACKGROUND

In related art, tissue harmonic imaging (THI) is widely used as a method for obtaining a B-mode image with higher spatial resolution than that of ordinary B-mode imaging. THI is a method of imaging using nonlinear components (for example, harmonic components such as second-order harmonic components) included in a reception signal.

In THI, various signal processing methods are performed, such as phase modulation (PM), amplitude modulation (AM), and AMPM being a combination of AM and PM. In PM, an ultrasonic wave is transmitted twice with the same amplitude and inverted phases in each scanning line, and two reception signals obtained thereby are added. By the addition processing, a signal is obtained in which fundamental wave components are canceled and second-order harmonic components generated in a second nonlinear propagation mainly remain. In PM, an image is obtained by imaging second-order harmonic components using the signal.

However, in an image generated by THI using the above signal processing methods, residual multiplex artifacts can occur as multiplex artifacts in some cases, due to mixing of a signal source of the reception signal from the previous transmission. Residual multiplex artifacts occur because a signal source of multiplexing is due to the previous transmission and multiplexing of fundamental wave components is left without being canceled. Multiplexing of fundamental wave components has a relatively low frequency, and a signal level thereof is higher than that of a harmonic signal. For this reason, multiplex residual artifacts may impede diagnosis using an image generated by THI.

### BRIEF DESCRIPTION OF THE DRAWINGS

FIG. 1 is a block diagram illustrating an example configuration of an ultrasonic diagnostic apparatus according to a first embodiment;

FIG. 2 is a block diagram illustrating an example configuration of a B-mode processor illustrated in FIG. 1;

FIG. 3A and FIG. 3B are diagrams for explaining THI;

FIG. 4 and FIG. 5 are diagrams for explaining residual multiplex artifacts occurring when THI is performed by multi focusing;

FIG. 6 is a diagram illustrating an ultrasonic transmission/reception order performed in a conventional method when THI is performed by multi focusing;

FIG. 7 is a diagram illustrating an ultrasonic transmission/reception order performed in a first embodiment when THI is performed by multi focusing;

FIG. 8 is a diagram for explaining a second embodiment;

FIG. 9 is a diagram for explaining a third embodiment;

FIG. 10, FIG. 11, FIG. 12, FIG. 13 and FIG. 14 are diagrams for explaining a scan sequence for removing a zeroth-order harmonic component;

FIG. 15 and FIG. 16 are diagrams for explaining a scan sequence for removing a zeroth-order harmonic component according to a fourth embodiment; and

FIG. 17A and FIG. 17B are diagrams for explaining a fifth embodiment.

### DETAILED DESCRIPTION

An ultrasonic diagnostic apparatus according to an embodiment comprises a transmitter/receiver, a signal processor, an image generator, and a controller. The transmitter/receiver executes ultrasonic transmission/reception sets a plurality of times on an identical scanning line by changing transmission conditions, and generates a plurality of sets of reception signals, the ultrasonic transmission/reception sets including a plurality of ultrasonic transmissions/receptions on the identical scanning line serving as a unit. The signal processor combines the plurality of the sets of the reception signals in each of the plurality of the sets, and generates a plurality of composite signals corresponding to each of the plurality of the sets. The image generator generates ultrasonic image data using the plurality of the composite signals. The controller controls an order of ultrasonic transmissions/receptions executed by the transmitter/receiver such that previous transmissions of respective transmissions corresponding to the plurality of the reception signals in one set combined by the signal processor have an identical transmission condition but different phase polarities.

Embodiments of an ultrasonic diagnostic apparatus will be explained hereinafter with reference to attached drawings.

### First Embodiment

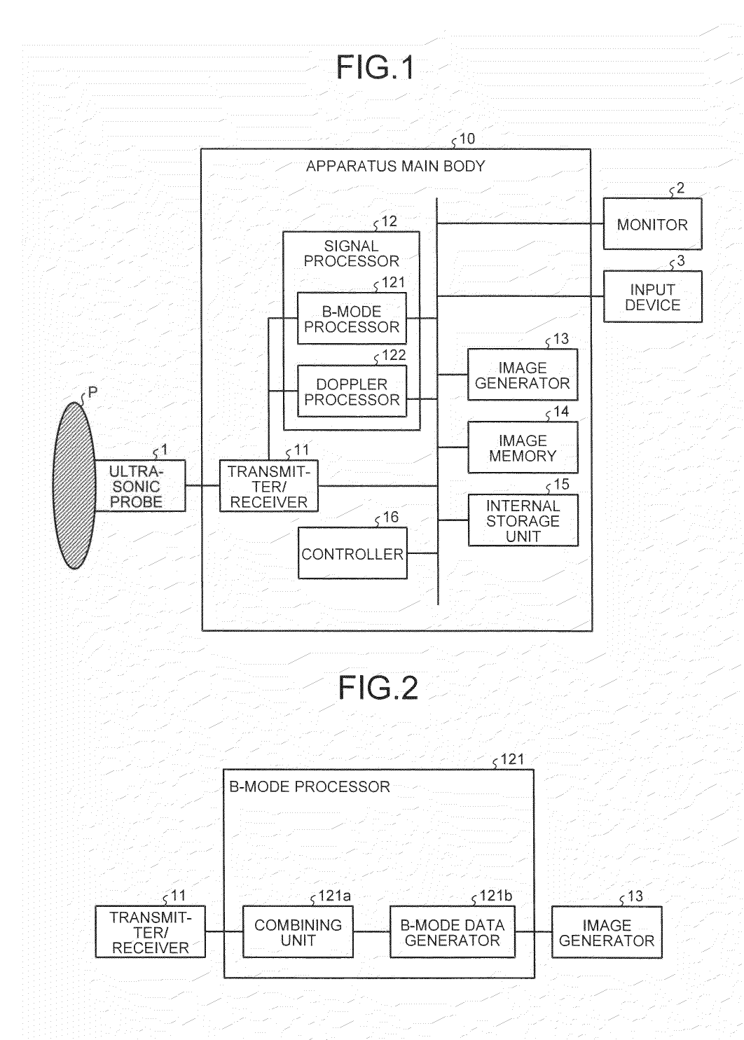

First, a configuration of an ultrasonic diagnostic apparatus according to a first embodiment will be explained hereinafter. FIG. 1 is a block diagram illustrating an example configuration of the ultrasonic diagnostic apparatus according to the first embodiment. As illustrated in FIG. 1, the ultrasonic diagnostic apparatus according to the first embodiment includes an ultrasonic probe 1, a monitor 2, an input device 3, and an apparatus main body 10.

The ultrasonic probe 1 includes a plurality of piezoelectric transducer elements. The piezoelectric transducer elements generate ultrasonic waves based on a driving signal supplied from a transmitter/receiver 11 included in the apparatus main body 10 described later. The piezoelectric transducer elements included in the ultrasonic probe 1 receive reflected waves from a subject P, and convert the reflected waves into electric signals (reflected wave signals). The ultrasonic probe 1 includes a matching layer provided in the piezoelectric transducer elements, and a backing material that prevents propagation of ultrasonic waves from the piezoelectric transducer elements to the rear. The ultrasonic probe 1 is detachably connected to the apparatus main body 10.

When an ultrasonic wave is transmitted from the ultrasonic probe 1 to the subject P, the transmitted ultrasonic wave is successively reflected by a discontinuous plane of acoustic impedance in a tissue of the subject P, received by the piezoelectric transducer elements included in the ultrasonic probe 1 as a reflected wave, and converted into a reflected wave signal. The amplitude of the reflected wave signal depends on a difference in acoustic impedance in the discontinuous plane that reflects the ultrasonic wave. When the transmitted ultrasonic pulse is reflected by a surface of a moving blood flow and a heart wall, the reflected wave signal is subjected to frequency shift depending on a velocity component of the moving object in an ultrasonic transmission direction by the Doppler effect.

The first embodiment is applicable to the case where the ultrasonic probe 1 is a 1D array probe that scans the subject P in a two-dimensional manner, and the case where the ultrasonic probe 1 is a mechanical 4D probe that scans the subject P in a three-dimensional manner or a 2D array probe.

The input device 3 includes a mouse, a keyboard, a button, a panel switch, a touch command screen, a foot switch, a trackball, and a joy stick and the like. The input device 3 receives various setting requests from the operator of the ultrasonic diagnostic apparatus, and transmits the received various setting requests to the apparatus main body 10.

The monitor 2 displays a graphical user interface (GUI) to enable the operator of the ultrasonic diagnostic apparatus to input various setting requests using the input device 3, and displays ultrasonic image data or the like generated in the apparatus main body 10.

The apparatus main body 10 is an apparatus that generates ultrasonic image data based on reflected waves received by the ultrasonic probe 1. The apparatus main body 10 illustrated in FIG. 1 is capable of generating two-dimensional ultrasonic image data based on a two-dimensional reflected wave signal, and capable of generating three-dimensional ultrasonic image data based on a three-dimensional reflected wave signal. However, the first embodiment is also applicable to the case where the apparatus main body 10 is dedicated to two-dimensional data.

As illustrated in FIG. 1, the apparatus main body 10 includes the transmitter/receiver 11, a signal processor 12, an image generator 13, an image memory 14, an internal storage unit 15, and a controller 16.

The transmitter/receiver 11 controls ultrasonic transmission/reception performed by the ultrasonic probe 1, based on instructions of the controller 16 described later. The transmitter/receiver 11 includes a pulse generator, a transmission delay unit, and a pulser and the like, and supplies a driving signal to the ultrasonic probe 1. The pulse generator repeatedly generates a rate pulse for forming a transmission ultrasonic wave at a predetermined pulse repetition frequency (PRF). The transmission delay unit focuses ultrasonic waves generated from the ultrasonic probe 1 into a beam, and provides each rate pulse generated by the pulse generator with a delay time for each piezoelectric transducer element necessary for determining the transmission directivity. The pulser applies a driving signal (driving pulse) to the ultrasonic probe 1 at a timing based on the rate pulse.

Specifically, the transmission delay unit changes the delay time to be supplied to each rate pulse, to adjust the transmission direction of the ultrasonic waves transmitted from the piezoelectric transducer element surface, as desired. The transmission delay unit changes the delay time to be supplied to each rate pulse, to also control the position of the focused point (transmission focus) of ultrasonic transmission in a depth direction. The transmitter/receiver 11 according to the first embodiment may be capable of performing multi focusing in which an ultrasonic beam is transmitted a plurality of times on the same scanning line at predetermined intervals at different depths of the transmission focus point. In the case of performing multi focusing, the transmission delay unit included in the transmitter/receiver 11 calculates a transmission delay time according to the depth of each transmission focus point, and supplies the transmission delay time to the pulser circuit.

The transmitter/receiver 11 has a function capable of instantaneously changing the transmission frequency and the transmission driving voltage or the like, to perform a predetermined scan sequence, based on instructions of the controller 16 described later. In particular, change of the transmission driving voltage is achieved by a transmission circuit of a linear amplifier type capable of instantaneously changing the value, or a mechanism that electrically switches a plurality of power source units.

The transmitter/receiver 11 includes an amplifier circuit, an analog/digital (A/D) converter, a reception delay circuit, an adder, and a quadrature detection circuit. The transmitter/receiver 11 performs various processing to a reflected wave signal received by the ultrasonic probe 1, and generates a reception signal (reflected wave data). The amplifier circuit amplifies the reflected wave signal for each channel, and performs gain correction. The A/D converter subjects the gain-corrected reflected wave signal to A/D conversion. The reception delay circuit provides the digital data with a reception delay time necessary for determining the reception directivity. The adder performs addition of the reflected wave signal provided with the reception delay time from the reception delay circuit. The addition performed by the adder emphasizes a reflected component in a direction according to the reception directivity of the reflected wave signal. Next, the quadrature detection circuit converts an output signal of the adder into an in-phase signal (I signal, I: In-phase) and a quadrature signal (Q signal, Q: Quadrature-phase) of the baseband. The quadrature detection circuit then stores the I signal and the Q signal (hereinafter referred to as the “IQ signal”) as a reception signal (reflected wave data) in a frame buffer (not illustrated).

The quadrature detection circuit may convert the output signal of the adder into a radio frequency (RF) signal, and store the RF signal in the frame buffer (not illustrated). The IQ signal and the RF signal are reception signals including phase information. The transmitter/receiver 11 is capable of performing parallel simultaneous reception in which reflected waves of a plurality of reception scanning lines are simultaneously received with a transmission ultrasonic wave provided on a transmission scanning line.

When the subject P is scanned in a two-dimensional manner, the transmitter/receiver 11 transmits a two-dimensional ultrasonic beam from the ultrasonic probe 1. The transmitter/receiver 11 generates two-dimensional reflected wave data from the two-dimensional reflected wave signal received by the ultrasonic probe 1. When the subject P is scanned in a three-dimensional manner, the transmitter/receiver 11 transmits a three-dimensional ultrasonic beam from the ultrasonic probe 1. The transmitter/receiver 11 then generates three-dimensional reflected wave data from the three-dimensional reflected wave signal received by the ultrasonic probe 1.

The signal processor 12 is a processor that performs various signal processing to a reception signal (reflected wave data) generated by the transmitter/receiver 11 from a reflected wave signal. As illustrated in FIG. 1, the signal processor 12 includes a B-mode processor 121 and a Doppler processor 122. The B-mode processor 121 receives a reception signal (reflected wave data) from the transmitter/receiver 11, performs logarithm amplification, envelope wave detection, and logarithm compression, and generates data (B-mode data) in which a signal intensity is expressed with brightness. The Doppler processor 122 performs frequency analysis of velocity information from the reception signal (reflected wave data) received from the transmitter/receiver 11, and generates data (Doppler data) obtained by extracting moving object information such as the velocity, dispersion, and power obtained by the Doppler effect for multiple points. The term “moving object” indicates, for example, a tissue such as a blood flow and a heart wall, and a contrast medium. The B-mode processor 121 and the Doppler processor 122 obtain a reception signal (reflected wave data) via the frame buffer described above.

The B-mode processor 121 and the Doppler processor 122 illustrated in FIG. 1 are capable of processing both two-dimensional reflected wave data and three-dimensional reflected wave data. Specifically, the B-mode processor 121 generates two-dimensional B-mode data from two-dimensional reflected wave data, and generates three-dimensional B-mode data from three-dimensional reflected wave data. The Doppler processor 122 generates two-dimensional Doppler data from two-dimensional reflected wave data, and generates three-dimensional Doppler data from three-dimensional reflected wave data. FIG. 2 is a block diagram illustrating an example configuration of the B-mode processor illustrated in FIG. 1.

As illustrated in FIG. 2, the B-mode processor 121 includes a combining unit 121a and a B-mode data generator 121b. The B-mode data generator 121b performs logarithm amplification, envelope wave detection, and logarithm compression to the reception signal (reflected wave data), to generate B-mode data. In the case where ordinary B-mode imaging is performed, no processing is performed by the combining unit 121a, and the B-mode data generator 121b generates B-mode data from the reception signal (reflected wave data) received from the transmitter/receiver 11.

In the case of performing harmonic imaging using phase modulation (PM), amplitude modulation (AM), or phase modulation amplitude modulation (AMPM), for example, the B-mode data generator 121b generates B-mode data from data (composite signal) that is output from the combining unit 121a. The processing performed by the combining unit 121a will be detailed later.

The image generator 13 generates ultrasonic image data from data generated by the signal processor 12 (the B-mode processor 121 and the Doppler processor 122). The image generator 13 generates two-dimensional B-mode image data, in which the intensity of the reflected wave is expressed with brightness, from the two-dimensional B-mode data generated by the B-mode processor 121. The image generator 13 also generates two-dimensional Doppler image data that indicates moving object information from the two-dimensional Doppler data generated by the Doppler processor 122. The two-dimensional Doppler image data is velocity image data, dispersion image data, power image data, or a combination thereof.

Generally, the image generator 13 converts (scan-converts) a scanning-line signal string of ultrasonic scanning into a scanning-line signal string of a video format represented by televisions or the like, to generate ultrasonic image data for display. Specifically, the image generator 13 performs coordinate transformation according to the ultrasonic scanning mode of the ultrasonic probe 1, to generate ultrasonic image data for display. The image generator 13 performs various image processing as well as scan conversion, such as image processing (smoothing) for regenerating a brightness average value image using a plurality of image frames having been subjected to scan conversion, and image processing (edge enhancement) using a differential filter in the image. In addition, the image generator 13 combines the ultrasonic image data with character information of various parameters, the divisions of a scale, and the body mark.

The B-mode data and the Doppler data are ultrasonic image data before scan conversion, and the data generated by the image generator 13 is ultrasonic image data for display that have been subjected to scan conversion. The B-mode data and the Doppler data are also referred to as “Raw Data”. The image generator 13 generates two-dimensional ultrasonic image data for display from two-dimensional ultrasonic image data before scan conversion.

In addition, the image generator 13 performs coordinate transformation to three-dimensional B-mode data generated by the B-mode processor 121, to generate three-dimensional B-mode image data. The image generator 13 also performs coordinate transformation to three-dimensional Doppler data generated by the Doppler processor 122, to generate three-dimensional Doppler image data. The image generator 13 generates the “three-dimensional B-mode image data and three-dimensional Doppler image data” as “three-dimensional ultrasonic image data (volume data)”.

In addition, the image generator 13 performs various rendering to the volume data, to generate two-dimensional image data to display the volume data on the monitor 2. The rendering performed by the image generator 13 includes, for example, processing for generating MPR image data from the volume data by performing multi planer reconstruction (MPR). The rendering performed by the image generator 13 includes, for example, volume rendering (VR) for generating two-dimensional image data reflecting three-dimensional information.

The image memory 14 is a memory that stores therein display image data generated by the image generator 13. The image memory 14 is also capable of storing data generated by the B-mode processor 121 and the Doppler processor 122. The B-mode data and the Doppler data stored in the image memory 14 can be called by the operator, for example, after diagnosis, and serve as display ultrasonic image data via the image generator 13. The image memory 14 can also store therein a reception signal (reflected wave data) that is output by the transmitter/receiver 11.

The internal storage unit 15 stores therein control programs for performing ultrasonic transmission/reception, image processing, and display processing, diagnostic information (such as patient's IDs, and doctor's observations and diagnosis), and various data such as diagnostic protocols and various body marks. The internal storage unit 15 is also used for storing image data stored in the image memory 14, if necessary. The data stored in the internal storage unit 15 can be transferred to an external device via an interface (not illustrated). The internal storage unit 15 can also store therein data transferred from an external device via an interface (not illustrated).

The controller 16 controls the whole processing of the ultrasonic diagnostic apparatus. Specifically, the controller 16 controls processing performed by the transmitter/receiver 11, the signal processor 12 (the B-mode processor 121 and the Doppler processor 122), and the image generator 13, based on various setting requests that are input by the operator via the input device 3 and various control programs and various data that are read from the internal storage unit 15. The controller 16 also performs control to display the display ultrasonic image data stored in the image memory 14 and the internal storage unit 15 on the monitor 2.

The transmitter/receiver 11 and other elements included in the apparatus main body 10 may be a software module program, although they may be formed of hardware such as an integrated circuit.

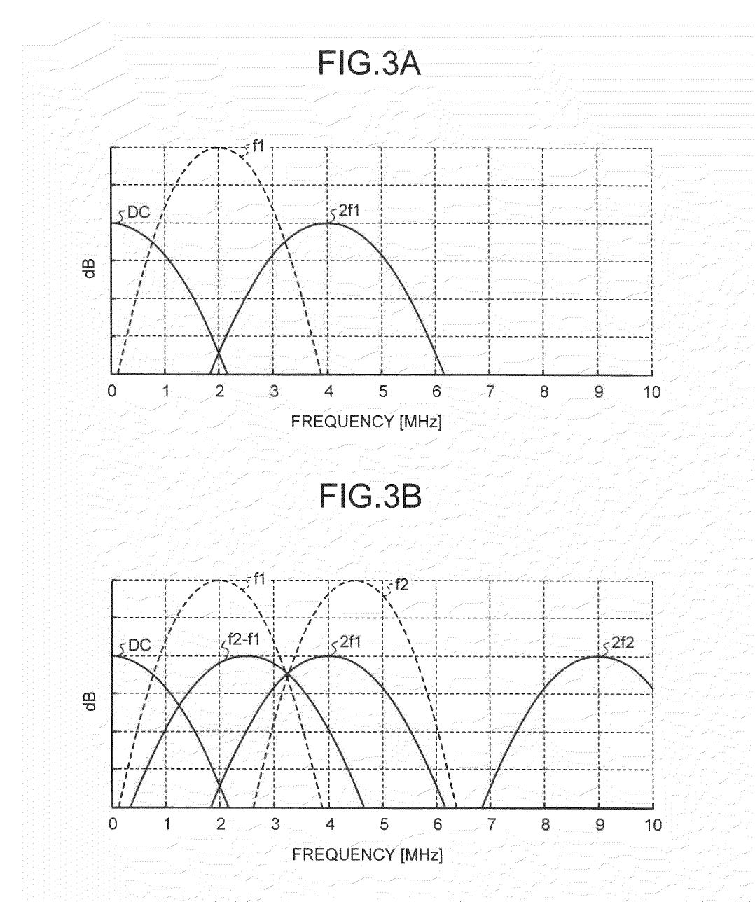

With the whole configuration of the ultrasonic diagnostic apparatus according to the first embodiment explained above, the ultrasonic diagnostic apparatus according to the first embodiment performs, for example, tissue harmonic imaging (THI) by PM also referred to as pulse inversion. As another example, the ultrasonic diagnostic apparatus according to the first embodiment performs THI by an imaging method (described later) using a difference sound component. FIG. 3A and FIG. 3B are diagrams for explaining THI. In FIG. 3A and FIG. 3B, the horizontal axis indicates frequency (unit: MHz), and the vertical axis indicates intensity of the reception signal (unit: dB).

For example, the transmitter/receiver 11 transmits a ultrasonic pulse of a fundamental wave having a central frequency “f1” twice in each scanning line with inverted phases, by a scan sequence that is set by the controller 16. Specifically, when the transmitter/receiver 11 transmits an ultrasonic wave having the central frequency “f1” twice in a scanning line, the transmitter/receiver 11 inverts the phase polarity of the first transmission ultrasonic wave to obtain the phase polarity of the second transmission ultrasonic wave. In this manner, the transmitter/receiver 11 generates two reception signals in a scanning line. The reception signal obtained by the first transmission “+1” is denoted by “r (+1)”, and the reception signal obtained by the second transmission “−1” is denoted by “r (−1)”.

In such a case, the polarities of the fundamental wave components derived from the fundamental wave are inverted between “r (+1)” and “r (−1)”. The polarities of second-order harmonic components derived from a second-order harmonic wave having a central frequency “2f” are the same between “r (+1)” and “r (−1)”. The combining unit 121a adds “r (+1)” to “r (−1)” to generate a composite signal. Because the signals “r (+1)” and “r (−1)” are IQ signals or RF signals having phase information, the addition performed by the combining unit 121a is coherent addition.

By the addition, the fundamental wave component (see “f1” illustrated in FIG. 3A) derived from the fundamental wave having the central frequency “f1” is canceled, and the second-order harmonic component (see “2f1” illustrated in FIG. 3A) derived from the second-order harmonic wave having the central frequency of “2f1” is doubled. Specifically, the composite signal is a harmonic signal in which the fundamental wave component is removed and the second-order harmonic component mainly remains. The B-mode data generator 121b generates B-mode data from a composite signal generated by the combining unit 121a, and the image generator 13 generates ultrasonic image data (B-mode image data) from the B-mode data. Imaging using a harmonic component is imaging using a center part of the ultrasonic beam. In addition, because side lobes have lower sound pressure than that of the main beam, harmonic waves are hardly generated. In view of the above, the lateral resolution of B-mode image data obtained by the above method is higher than that of ordinary B-mode image data.

However, because the bandwidth of a harmonic component is narrow, or penetration in a deep region is insufficient due to harmonic wave reception, the above method can fail to improve axial resolution. In recent years, as THI for obtaining B-mode image data with high lateral resolution and axial resolution, a method has been put to practical use in which imaging is performed using a second-order harmonic component and a difference sound component included in the reception signal. In an imaging method using a difference sound component, an ultrasonic pulse having a composite waveform obtained by mixing two fundamental waves at different central frequencies is transmitted a plurality of times with phase inversion in each scanning line, and reception signals thereof are combined.

For example, suppose that the two fundamental waves used in the imaging method using a difference sound component are a first fundamental wave having a central frequency “f1” and a second fundamental wave having a central frequency “f2” that is larger than “f1”. The transmitter/receiver 11 transmits an ultrasonic pulse having a composite waveform obtained by combining the waveform of the first fundamental wave with the waveform of the second fundamental wave from the ultrasonic probe 1. The composite waveform is a waveform obtained by combining the waveform of the first fundamental wave with the waveform of the second fundamental waveform with mutual phases adjusted such that a difference sound component having the same polarity as that of the second-order harmonic component is generated. The controller 16 adjusts the phase condition. The phase condition for generating a difference sound component having the same polarity as that of the second-order harmonic component will be referred to as the “same polarity phase condition”.

As illustrated in FIG. 3B, the reception signal obtained by the transmission ultrasonic wave having the composite waveform of the first fundamental wave and the second fundamental wave includes the first fundamental wave component derived from the first fundamental wave with the central frequency “f1”, and the second fundamental wave component derived from the second fundamental wave with the central frequency “f2”. As illustrated in FIG. 3B, the reception signal also includes a second-order harmonic component derived from the second-order harmonic wave with the central frequency “2f1”, and a second-order harmonic component derived from the second-order harmonic wave with the central frequency “2f2”. In the case of using two fundamental waves having different central frequencies, the reception signal includes a difference sound component derived from a difference sound “f2−f1” between the second fundamental wave and the first fundamental wave, as illustrated in FIG. 3B. Although it is not illustrated in FIG. 3B, the reception signal also includes an addition sound component derived from an addition sound “f1+f2” of the second fundamental wave and the first fundamental wave.

The transmitter/receiver 11 transmits the transmission ultrasonic wave having a composite waveform a plurality of times (for example, twice) with inverted phases. For example, when the transmitter/receiver 11 transmits the transmission ultrasonic wave having a composite waveform twice in a scanning line, the transmitter/receiver 11 inverts the phase polarity of the first transmission ultrasonic wave to obtain the phase polarity of the second transmission ultrasonic wave. The transmitter/receiver 11 thus generates two reflected wave data items in one scanning line. The reflected wave data item obtained by the first transmission “+1” is referred to as “R (+1)”, and the reflected wave data item obtained by the second transmission “−1” is referred to as “R (−1)”.

In such a case, the polarity of the first fundamental wave component and the polarity of the second fundamental wave component are the opposite, between “R (+1)” and “R (−1)”. The polarity of the second-order harmonic component derived from the second-order harmonic wave “2f1”, the polarity of the second-order harmonic component derived from the second-order harmonic wave “2f2”, and the polarity of the difference sound component derived from the difference sound “f2−f1” are the same between “R (+1)” and “R (−1)”. The combining unit 121a adds (coherent addition) “R (+1)” to “R (−1)”, to generate a composite signal. The composite signal is a harmonic signal in which the fundamental wave components are removed, and the difference sound component and the second-order harmonic components mainly remain.

The combining unit 121a removes the second-order harmonic component derived from the second-order harmonic wave “2f2” from the composite signal (composite data) by filtering. As another example, for example, the controller 16 sets the frequency band of the second-order harmonic component derived from the second-order harmonic wave “2f2” to a band that falls out of the frequency band that can be received by the ultrasonic probe 1. The combining unit 121a thus generates a composite signal (composite harmonic signal) in which the difference sound component of “f2−f1” and the second-order harmonic component of “2f1” are extracted.

B-mode data is generated thereafter from the composite data that is output by the combining unit 121a, and the image generator 13 generates ultrasonic image data (B-mode image data) from the B-mode data. The composite data that is output by the combining unit 121a is a composite harmonic signal including the second-order harmonic component at the low frequency side and the difference sound component, and serves as a harmonic echo having a broader band than that of a signal obtained by conventional THI. In an imaging method using a difference sound component, imaging using the composite harmonic signal produces B-mode image data having high spatial resolution (lateral resolution and axial resolution).

In the imaging method using a difference sound component, the controller 16 adjusts the values of “f1” and “f2” in accordance with the frequency band to be imaged. For example, in the case of “f1=f” and performing imaging at a broad frequency band having “2f” as the center, the value of “f2” is adjusted to “f2=3×f”. In addition, for example, in the case of “f1=f” and performing imaging at a broad frequency band having a central frequency higher than “2f”, the value of “f2” is adjusted to a value greater than “3×f”, such as “f2=3.5×f”. As another example, in the case of “f1=f” and performing imaging at a broad frequency band having a central frequency lower than “2f”, the value of “f2” is adjusted to a value smaller than “3×f”, such as “f2=2.5×f”.

In the meantime, harmonic components generated in a second nonlinear propagation include zeroth-order harmonic components, as well as harmonic components (such as second-order harmonic components) to be imaged. A zeroth-order harmonic component is a harmonic component at a low band having a direct current as the center, and also referred to as a DC harmonic component. In FIG. 3A and FIG. 3B, a zeroth-order harmonic component is schematically illustrated as “DC”. The component “DC” illustrated in FIG. 3A and FIG. 3B is a component corresponding to the term “zero-order” among nonlinear components (harmonic components) of the reception signal.

However, images generated by THI using the above signal processing method may include a residual multiplex artifact as a multiplex artifact that is caused by mixing of a signal source of the reception signal from the previous transmission.

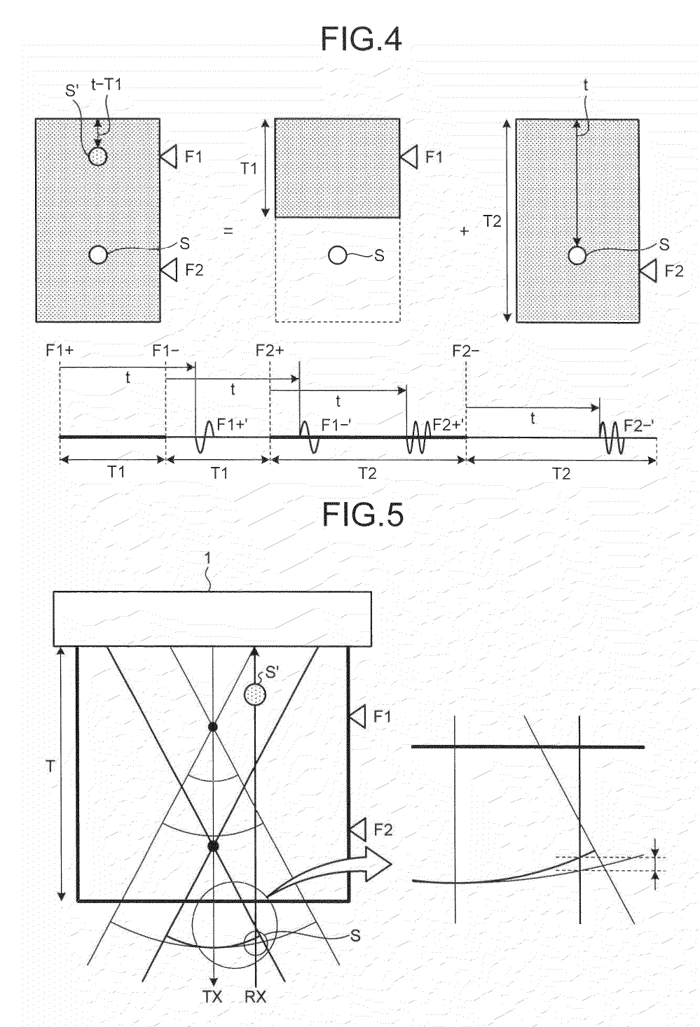

The residual multiplex artifact is caused because, since the multiple signal source originates from the previous transmission, multiplex of the fundamental wave components is left without being canceled, due to “shift of the transmission wave surface caused by difference in position of the transmission scanning line”, “difference in transmission focus position when multi focusing is performed”, and “difference in PRF (pulse repetition frequency)”. For example, residual multiplex artifacts occur when THI using PM (pulse inversion) or THI using a difference sound component is performed in combination with multi focusing. FIG. 4 and FIG. 5 are diagrams for explaining residual multiplex artifacts occurring when THI is performed by multi focusing.

FIG. 4 illustrates the case where residual multiplex artifacts occur due to difference in PRF between focuses when multi focusing is performed. In FIG. 4, “F1” denotes the position of the transmission focus that is set in a shallow part, and “F2” denotes the position of the transmission focus that is set in a deep part. FIG. 4 illustrates that ultrasonic transmission/reception for “F1” is performed at “pulse interval: T1”, and the visual field depth at “transmission focus: f1” is “in-vivo sound velocity×T1”. FIG. 4 also illustrates that ultrasonic transmission/reception for “F2” is performed at “pulse interval: T2”, and the visual field depth at “transmission focus: F2” is “in-vivo sound velocity×T2”. Specifically, in the example illustrated in FIG. 4, ultrasonic transmission/reception for “F1” is performed at “PRF: 1/T1”, and ultrasonic transmission/reception for “F2” is performed at “PRF: 1/T2”.

In addition, in FIG. 4, “F1+” denotes the transmission waveform of an ultrasonic pulse that is transmitted at the first time with the focus on the position of “transmission focus: F1”, and “F1−” denotes the transmission waveform of an ultrasonic pulse that is transmitted at the second time with the phase polarity inverted from “F1+”. In FIG. 4, “F2+” denotes the transmission waveform of an ultrasonic pulse that is transmitted at the first time with the focus on the position of “transmission focus: F2”, and “F2−” denotes the transmission waveform of an ultrasonic pulse that is transmitted at the second time with the phase polarity inverted from “F2+”.

In such a case, as illustrated in the lower drawing of FIG. 4, “F1+” is transmitted at the first time, and a reflected wave originated from “F1+” is received until T1 has elapsed. Next, as illustrated in the lower drawing of FIG. 4, “F1−” is transmitted at the second time, and a reflected wave originated from “F1+” is received until T1 has elapsed. Next, as illustrated in the lower drawing of FIG. 4, “F2+” is transmitted at the third time, and a reflected wave originated from “F2+” is received until T2 has elapsed. Next, as illustrated in the lower drawing of FIG. 4, “F2−” is transmitted at the fourth time, and a reflected wave originated from “F2+” is received until T12 has elapsed. The above four transmission waveforms may be transmission waveforms based on PM, or transmission waveforms based on an imaging method using a difference sound component.

The transmitter/receiver 11 generates a reception signal based on the first transmission and a reception signal based on the second transmission. The combining unit 121a adds the reception signal obtained by the first transmission to the reception signal obtained by the second transmission, to generate a composite signal with the visual field depth up to “in-vivo sound velocity×T1” on the corresponding scanning line. The transmitter/receiver 11 also generates a reception signal based on the third transmission and a reception signal based on the fourth transmission. The combining unit 121a adds the reception signal obtained by the third transmission to the reception signal obtained by the fourth transmission, to generate a composite signal with the visual field depth up to “in-vivo sound velocity×T2” on the corresponding scanning line. Such processing is performed in each of the scanning lines that form the scanning range.

The above processing produces B-mode image data with “transmission focus: F1” and B-mode image data with “transmission focus: F2”. Under the control of the controller 16, the image generator 13 generates image data obtained by combining the B-mode image data with “transmission focus: F1” with the B-mode image data with “transmission focus: F2”, and the monitor 2 displays the image data.

FIG. 4 illustrates that a strong reflector S exists at the position of “in-vivo sound velocity×t” between “in-vivo sound velocity×T1” and “in-vivo sound velocity×T2”, in the depth direction of a scanning line. In such a case, as illustrated in FIG. 4, a reflected wave “F2+′” from the strong reflector S with “F2+” is received after “t” has elapsed since start of the third transmission, that is, during the third transmission/reception period. In addition, a reflected wave “F2−′” from the strong reflector S with “F2−” is received after “t” has elapsed since start of the fourth transmission, that is, during the fourth transmission/reception period. The fundamental wave component of “F2+′” and the fundamental wave component of “F2−′” are canceled by addition of the reception signal obtained by the third transmission to the reception signal obtained by the fourth transmission.

However, as illustrated in FIG. 4, a reflected wave “F1+′” from the strong reflector S with “F1+” is received after “t−T1” has elapsed since start of the second transmission after the end of the first transmission/reception. In addition, as illustrated in FIG. 4, a reflected wave “F2+′” from the strong reflector S with “F1−” is received after “t−T1” has elapsed since the start of the third transmission after end of the second transmission/reception.

Therefore, the fundamental wave component of “F1+′” is not removed by adding the reception signal obtained by the first transmission to the reception signal obtained by the second transmission, and is left without being removed. In addition, the fundamental wave component of “F1−′” is not removed by adding the reception signal obtained by the third transmission to the reception signal obtained by the fourth transmission, but is left without being removed.

Consequently, as illustrated in FIG. 4, residual multiplex S′ appears at a position of the depth corresponding to “t−T1” in the B-mode image data. However, residual multiplex S′ does not appear in the case of “t<T1<T2” or “t<T1=T2”, although it appears in the case of “T1<t<T2”.

However, residual multiplex artifacts may occur even when multi focusing is performed at sufficiently long pulse intervals. For example, residual multiplex artifacts occur when THI using multi focusing is performed together with parallel simultaneous reception to improve the frame rate. FIG. 5 illustrates the case where a residual multiplex artifact is caused by difference in arrival time between reflected wave signals due to shift in transmission wave surface, when employing both multi focusing and parallel simultaneous reception.

FIG. 5 illustrates the case where an ultrasonic wave is transmitted on a transmission scanning line TX from the ultrasonic probe 1, and a reflected wave is received on a reception scanning line RX located at a position distant from the transmission scanning line TX at parallel simultaneous reception. FIG. 5 also illustrates the case where the pulse interval at each transmission is “T”. FIG. 5 illustrates the case where the strong reflector S is located at a position deeper than “in-vivo sound velocity×T” on the reception scanning line RX.

Specifically, in FIG. 5, performed are the first transmission/reception with “F1+”, the second transmission/reception with “F1−”, the third transmission/reception with “F2+”, and the fourth transmission/reception with “F2−” at intervals “T”. As illustrated in FIG. 5, at the position where the strong reflector S is located, “phase shift” occurs between the transmission wave surfaces of “F1+” and “F1−” and the transmission wave surfaces of “F2+” and “F2−”, due to shift in position of the transmission focus. Due to the phase shift, the arrival time of the reflected wave signal of the strong reflector S at “transmission focus: F1” is different from the arrival time of the reflected wave signal of the strong reflector S at “transmission focus: F2”. In this manner, a residual multiplex S′ occurs on the reception scanning line RX, as illustrated in FIG. 5. Even when the strong reflector S is located at a position shallower than “in-vivo sound velocity×T”, the residual multiplex S′ occurs due to the difference in arrival time between the reflected wave signals due to shift in transmission wave surface. The residual multiplex S′ caused by difference in transmission wave surface occurs, also when PRF is different between “transmission focus: F1” and “transmission focus: F2”.

In addition, residual multiplex caused by the fourth transmission of a scanning line adjacent to the scanning line is mixed into the first transmission/reception period in the scanning line. In such a case, the fundamental wave component of the residual multiplex is left without being removed, due to shift in transmission wave surface caused by difference in transmission scanning line.

The fundamental wave component of residual multiplex has a relatively low frequency, and has a signal level higher than that of a harmonic signal. For this reason, multiple residual artifacts may obstruct diagnosis using an image generated by THI.

Therefore, according to the first embodiment, the following processing is performed to remove residual multiplex artifacts.

First, the transmitter/receiver 11 executes ultrasonic transmission/reception sets a plurality of times on an identical scanning line by changing transmission conditions, and generates a plurality of sets of reception signals, the ultrasonic transmission/reception set including a plurality of ultrasonic transmissions/receptions on the identical scanning line serving as a unit. The signal processor 12 (combining unit 121a) combines the reception signals in each of the plurality of the sets, and generates a plurality of composite signals corresponding to the sets. Specifically, the signal processor 12 (combining unit 121a) combines the plurality of the reception signals of the sets, and generates a plurality of composite signals corresponding to each of the plurality of the sets. The image generator 13 generates ultrasonic image data using the plurality of the composite signals. Specifically, the B-mode data generator 121b generates B-mode data from the composite signals of the scanning lines, and the image generator 13 generates B-mode image data from the B-mode data. The controller 16 controls the order of ultrasonic transmissions/receptions executed by the transmitter/receiver 11 such that previous transmissions of respective transmissions corresponding to the plurality of the reception signals in one set combined by the signal processor 12 (combining unit 121a) have an identical transmission condition but different phase polarities.

The following is an example of processing executed with restriction on the transmission order. The transmitter/receiver 11 causes the ultrasonic probe 1 to transmit a first ultrasonic pulse based on a first transmission condition relating to a certain scanning line. The transmitter/receiver 11 then causes the ultrasonic probe 1 to transmit, subsequent to the first ultrasonic pulse, a second ultrasonic pulse based on a second transmission condition relating to the certain scanning line and being different from the first transmission condition. Subsequently, the transmitter/receiver 11 causes the ultrasonic probe 1 to transmit, after the second ultrasonic pulse, a third ultrasonic pulse based on a third transmission condition relating to the certain scanning line and including a phase polarity different from that under the first transmission condition. The transmitter/receiver 11 then causes the ultrasonic probe 1 to transmit, subsequent to the third ultrasonic pulse, a fourth ultrasonic pulse based on a fourth transmission condition relating to the certain scanning line and including a phase polarity different from that under the second transmission condition. The transmitter/receiver 11 generates a first reception signal based on a reflected wave received by the ultrasonic probe 1 as a result of transmission of the first ultrasonic pulse. The transmitter/receiver 11 also generates a second reception signal based on a reflected wave received by the ultrasonic probe 1 as a result of transmission of the second ultrasonic pulse. The transmitter/receiver 11 also generates a third reception signal based on a reflected wave received by the ultrasonic probe 1 as a result of transmission of the third ultrasonic pulse. The transmitter/receiver 11 also generates a fourth reception signal based on a reflected wave received by the ultrasonic probe as a result of transmission of the fourth ultrasonic pulse. The signal processor 12 (combining unit 121a) generates a first composite signal by combining the first reception signal with the third reception signal, and generates a second composite signal by combining the second reception signal with the fourth reception signal. The image generator 13 generates image data based on the first composite signal and the second composite signal.

The transmission conditions transmitter/receiver 11 changes at each of the plurality of the sets are at least one of a transmission focus position, a transmission frequency, and a transmission waveform. In an example of the processing performed with restriction on the transmission order, the transmitter/receiver 11 changes at least one of transmission conditions including the transmission focus position, the transmission frequency, and the transmission waveform between the first transmission condition and the third transmission condition and between the second transmission condition and the fourth transmission condition In the first embodiment, the transmitter/receiver 11 executes the ultrasonic transmission/reception sets a plurality of times on the scanning line at different transmission focus positions, the ultrasonic transmission/reception sets including two ultrasonic transmissions/receptions serving as a unit and being executed twice on the identical scanning line with inverted phase polarities. Specifically, in the first embodiment, the transmitter/receiver 11 executes the ultrasonic transmission/reception set, for the purpose of imaging harmonic components (such as second-order harmonic components, and difference sound components and second-order harmonic components), a plurality of times on the scanning line at different transmission focus positions. The transmitter/receiver 11 then generates reception signals for the sets.

For example, in the first embodiment, the transmitter/receiver 11 executes two sets of ultrasonic transmission/reception at different transmission focus positions on the scanning line, and each of the sets includes two ultrasonic transmissions/receptions executed with inverted phase polarities on the scanning line. The transmitter/receiver 11 then generates four reception signals.

In the first embodiment, the signal processor 12 (combining unit 121a) adds two reception signals in each of the sets, and generates a plurality of composite signals on the scanning line. Specifically, the signal processor 12 (combining unit 121a) adds the two reception signals of each of the plurality of the sets, and generates a composite signal for each transmission focus position in each scanning line. For example, the combining unit 121a adds the reception signal originated from “F1+” to the reception signal originated from “F1−”, and generates a composite signal of “transmission focus: F1”. For example, the combining unit 121a adds the reception signal originated from “F2+” to the reception signal originated from “F2−”, and generates a composite signal of “transmission focus: F2”.

In the first embodiment, the image generator 13 generates ultrasonic image data at each transmission focus position from the composite signal at each transmission focus position.

The controller 16 controls the order of ultrasonic transmissions/receptions executed by the transmitter/receiver 11, as described above. The controller 16 controls the order of the ultrasonic transmissions/receptions executed by the transmitter/receiver 11 such that previous transmissions of respective transmissions corresponding to the plurality of the reception signals in one set combined by the signal processor 12 (combining unit 121a) have an identical transmission condition but different phase polarities. In the first embodiment, the controller 16 controls the transmission order such that previous transmissions of two respective transmissions corresponding to the two reception signals in one set added by the signal processor 12 (combining unit 121a) have an identical transmission focus position.

In the example of processing executed with restriction on the transmission order, the transmitter/receiver 11 inverts the phase polarity of the first ultrasonic pulse to obtain the phase polarity of the third ultrasonic pulse, with transmission focus positions of the first ultrasonic pulse and the third ultrasonic pulse serving as a first position “F1”, and inverts the phase polarity of the second ultrasonic pulse to obtain the phase polarity of the fourth ultrasonic pulse, with transmission focus positions of the second ultrasonic pulse and the fourth ultrasonic pulse serving as a second position “F2” different from the first position. The signal processor 12 (combining unit 121a) generates a first composite signal by adding the first reception signal to the third reception signal, and generates a second composite signal by adding the second reception signal to the fourth reception signal.

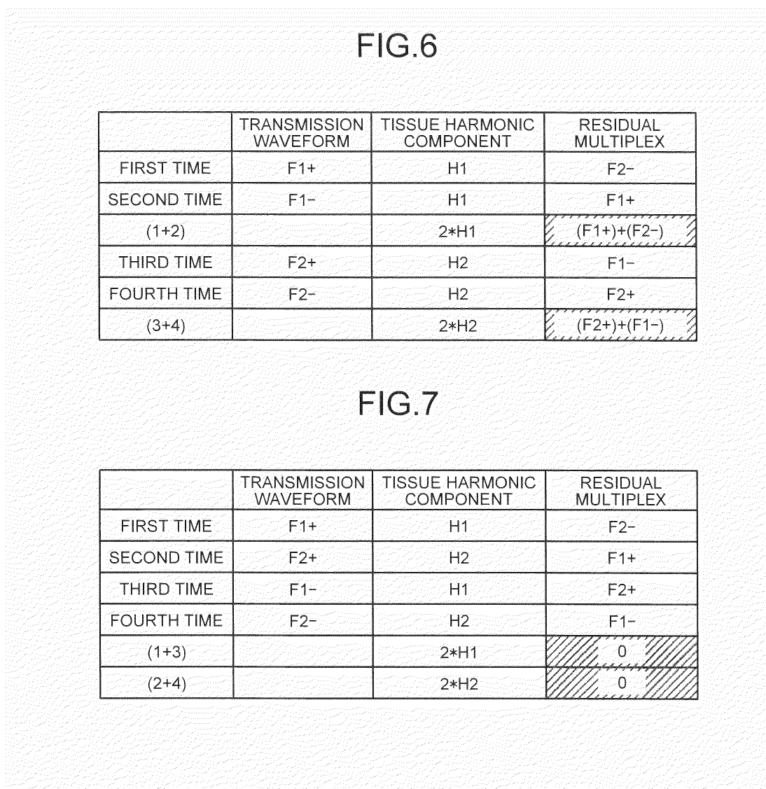

FIG. 6 is a diagram illustrating an ultrasonic transmission/reception order performed in a conventional method, when performing multi focus execution of THI. FIG. 7 is a diagram illustrating an ultrasonic transmission/reception order performed in the first embodiment when THI is performed by multi focusing.

As illustrated in FIG. 6, in conventional art, transmission/reception with an ultrasonic pulse having a transmission waveform “F1+” is performed at the first time, and transmission/reception with an ultrasonic pulse having a transmission waveform “F1−” is performed at the second time. In addition, as illustrated in FIG. 6, in conventional art, transmission/reception with an ultrasonic pulse having a transmission waveform “F2+” is performed at the third time, and transmission/reception with an ultrasonic pulse having a transmission waveform “F2−” is performed at the fourth time.

Subsequently, addition processing “1+2” is performed to add a reception signal of the first time to a reception signal of the second time. This addition produces a composite signal obtained by extracting “2*H1” obtained by adding “tissue harmonic component: H1” of the reception signal of the first time to “tissue harmonic component: H1” of the reception signal of the second time, as illustrated in FIG. 6. In addition, addition processing “3+4” is performed to add a reception signal of the third time to a reception signal of the fourth time. This addition produces a composite signal obtained by extracting “2*H2” obtained by adding “tissue harmonic component: H2” of the reception signal of the third time to “tissue harmonic component: H2” of the reception signal of the fourth time, as illustrated in FIG. 6.

However, in conventional art, the residual multiplex “F2−” of the fourth time on the adjacent scanning line is mixed into the reception signal of the first time, as illustrated in FIG. 6. In addition, in conventional art, the residual multiplex “F1+” of the first time on the same scanning line is mixed into the reception signal of the second time, as illustrated in FIG. 6. For this reason, in conventional art, “(F1+)+(F2−)” is left without being removed in the composite signal obtained by the addition “1+2”, as illustrated in FIG. 6.

In addition, in conventional art, the residual multiplex “F1−” of the second time on the same scanning line is mixed into the reception signal of the third time, as illustrated in FIG. 6. In addition, in conventional art, the residual multiplex “F2+” of the third time on the same scanning line is mixed into the reception signal of the fourth time, as illustrated in FIG. 6. For this reason, in conventional art, “(F2+)+(F1−)” is left without being removed in the composite signal obtained by the addition “3+4”, as illustrated in FIG. 6.

In contrast, in the first embodiment, transmission/reception with an ultrasonic pulse (first ultrasonic pulse) having the transmission waveform “F1+” is performed at the first time, and transmission/reception with an ultrasonic pulse (second ultrasonic pulse) having the transmission waveform “F2+” is performed at the second time as illustrated in FIG. 7. In addition, in the first embodiment, transmission/reception with an ultrasonic pulse (third ultrasonic pulse) having the transmission waveform “F1−” is performed at the third time, and transmission/reception with an ultrasonic pulse (fourth ultrasonic pulse) having the transmission waveform “F2−” is performed at the fourth time as illustrated in FIG. 7.

Specifically, in the first embodiment, the second transmission performed in the conventional method is changed to the third transmission, and the third transmission performed in the conventional method is changed to the second transmission. In addition, in the first embodiment, addition processing “1+3” is performed to add a reception signal (first reception signal) of the first time to a reception signal (third reception signal) of the third time. This addition produces a composite signal (first composite signal) in which “tissue harmonic component: 2*H1” is extracted, as illustrated in FIG. 7. In addition, in the first embodiment, addition processing “2+4” is performed to add a reception signal (second reception signal) of the second time to a reception signal (fourth reception signal) of the fourth time. This addition produces a composite signal (second composite signal) in which “tissue harmonic component: 2*H2” is extracted, as illustrated in FIG. 7.

By the above restriction on the transmission order, in the addition “1+3” of the first embodiment, the transmission focus position of the fourth time serving as the previous transmission of the first time is the same as the transmission focus position of the second time serving as the previous transmission of the third time, that is, “F2”. In addition, by the above restriction on the transmission order, in the addition “2+4” of the first embodiment, the transmission focus position of the first time serving as the previous transmission of the second time is the same as the transmission focus position of the third time serving as the previous transmission of the fourth time, that is, “F1”.

By the above restriction on the transmission order, in the first embodiment, residual multiplex “F2−” of the fourth time on the adjacent scanning line is mixed into the reception signal of the first time, as illustrated in FIG. 7. In addition, in the first embodiment, the residual multiplex “F1+” of the first time on the same scanning line is mixed into the reception signal of the second time, as illustrated in FIG. 7. In addition, in the first embodiment, the residual multiplex “F2+” of the second time on the same scanning line is mixed into the reception signal of the third time, as illustrated in FIG. 7. In addition, in the first embodiment, the residual multiplex “F1−” of the third time on the same scanning line is mixed into the reception signal of the fourth time, as illustrated in FIG. 7.

However, in the first embodiment, the addition “1+3” cancels the residual multiplex “F2−” and the residual multiplex “F2+”, and the residual multiplex of the composite signal (first composite signal) is “0”, as illustrated in FIG. 7. In addition, in the first embodiment, the addition “2+4” cancels the residual multiplex “F1+” and the residual multiplex “F1−”, and the residual multiplex of the composite signal (second composite signal) is “0”, as illustrated in FIG. 7.

As described above, in the first embodiment, by the above restriction condition for the transmission order, the fundamental wave components of residual multiplexes included in the two respective reception signals to be added have inverted phase polarities, and have no phase shift because they are caused by the transmission waveforms at the same transmission focus position. In this manner, according to the first embodiment, under the above restriction condition for the transmission order, residual multiplex artifacts can be removed when THI using PM or THI using a difference sound component is performed by multi focusing, even when PRF differs for each transmission focus position or parallel simultaneous reception is performed.

### Second Embodiment

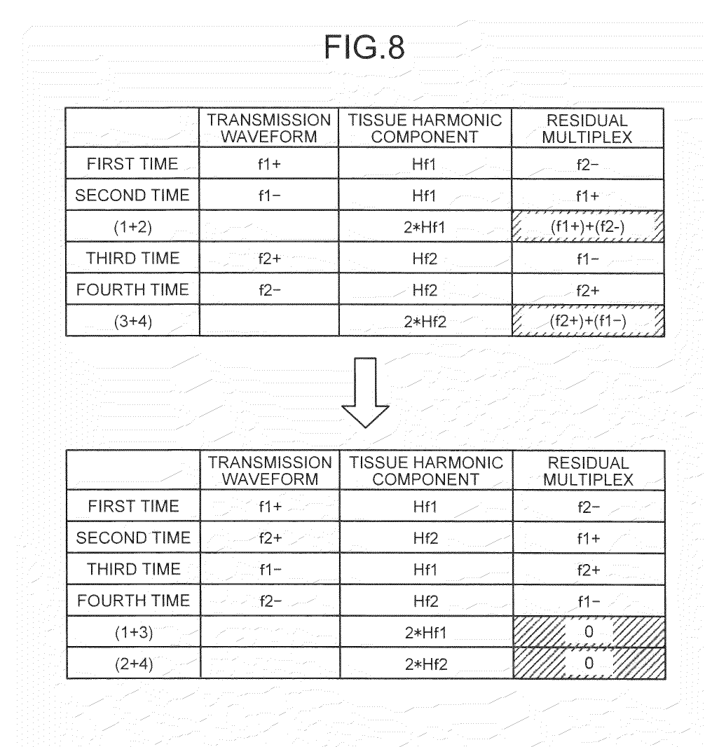

In a second embodiment, a restriction condition for the transmission order that is different from that of the first embodiment will be explained with reference to FIG. 8. FIG. 8 is a diagram for explaining the second embodiment.

In THI using PM or THI using a difference sound component, there are also the cases where ultrasonic transmissions are performed at different transmission frequencies, to broaden the band of the harmonic component to be imaged, and to obtain image data with high image quality.

In such a case, the transmitter/receiver 11 performs the following processing for the purpose of imaging a harmonic component as a certain signal component. The transmitter/receiver 11 executes the ultrasonic transmission/reception sets a plurality of times on the scanning line with different transmission frequencies, the ultrasonic transmission/reception sets including two ultrasonic transmissions/receptions serving as a unit and being executed twice on the identical scanning line with inverted phase polarities. In the above case, the transmission condition the transmitter/receiver 11 changes at each of the plurality of the sets is a transmission frequency. The transmitter/receiver 11 adds two reception signals in each of the plurality of the sets thereafter, to generate a plurality of composite signals on the scanning line. The image generator 13 generates an ultrasonic image data group using each of the sets of composite signals, and generates image data obtained by combining the ultrasonic image data group as the ultrasonic image data.

For example, the transmitter/receiver 11 executes a first set including ultrasonic transmission/reception with the transmission waveform “f1+” having the central frequency “f1” and ultrasonic transmission/reception with the transmission waveform “f1−” having a phase polarity inverted from that of the transmission waveform “f1+”, on the same scanning line. In addition, for example, the transmitter/receiver 11 executes a second set including ultrasonic transmission/reception with the transmission waveform “f2+” having the central frequency “f2” and ultrasonic transmission/reception with the transmission waveform “f2−” having a phase polarity inverted from that of the transmission waveform “f2+”, on the same scanning line. The transmitter/receiver 11 then generates a reception signal of “f1+”, a reception signal of “f1−”, a reception signal of “f2+”, and a reception signal of “f2−” on each scanning line.

The combining unit 121a adds the reception signal of “f1+” including “tissue harmonic component: Hf1” to the reception signal of “f1−” including “tissue harmonic component: Hf1”, to generate a composite signal in which “tissue harmonic component 2*Hf1” is extracted. In addition, the combining unit 121a adds the reception signal of “f2+” including “tissue harmonic component: Hf2” to the reception signal of “f2−” including “tissue harmonic component: Hf2”, to generate a composite signal in which “tissue harmonic component 2*Hf2” is extracted.

The image generator 13 generates B-mode image data from B-mode data generated from the composite signal including the extracted “tissue harmonic component: 2*Hf1”. The image generator 13 also generates B-mode image data from B-mode data generated from the composite signal including the extracted “tissue harmonic component: 2*Hf2”. Subsequently, for example, the image generator 13 performs arithmetic mean operation to the two B-mode image data items, to generate B-mode image data with high image quality obtained by imaging “tissue harmonic component: 2*Hf1+2*Hf2”.

In conventional art, as illustrated in the upper drawing of FIG. 8, the first transmission is performed with the transmission waveform “f1+”, the second transmission is performed with the transmission waveform “f1−”, the third transmission is performed with the transmission waveform “f2+”, and the fourth transmission is performed with the transmission waveform “f2−”.

However, in conventional art, the residual multiplex “f2−” of the fourth time on the adjacent scanning line is mixed into the reception signal obtained by the first transmission, as illustrated in the upper drawing of FIG. 8. In addition, in conventional art, the residual multiplex “f1+” of the first time on the same scanning line is mixed into the reception signal obtained by the second transmission, as illustrated in the upper drawing of FIG. 8. For this reason, in conventional art, “(f1+)+(f2−)” is left without being removed in the composite signal obtained by the addition “1+2”, as illustrated in the upper drawing of FIG. 8.

In addition, in conventional art, the residual multiplex “f1−” of the second time on the same scanning line is mixed into the reception signal obtained by the third transmission, as illustrated in the upper drawing of FIG. 8. In addition, in conventional art, the residual multiplex “f2+” of the third time on the same scanning line is mixed into the reception signal obtained by the fourth transmission, as illustrated in the upper drawing of FIG. 8. For this reason, in conventional art, “(f2+)+(f1−)” is left without being removed in the composite signal obtained by the addition “3+4”, as illustrated in the upper drawing of FIG. 8.

In contrast, the controller 16 according to the second embodiment restricts the transmission order such that previous transmissions of two respective transmissions corresponding to the two reception signals in one set added by the combining unit 121a have an identical transmission frequency. The above addition serves as processing to extract a harmonic component.

In the example executed with the restriction on the transmission order, the transmitter/receiver 11 causes the ultrasonic probe 1 to execute ultrasonic transmissions in the order of the first ultrasonic pulse, the second ultrasonic pulse, the third ultrasonic pulse, and the fourth ultrasonic pulse. The transmitter/receiver 11 inverts the phase polarity of the first ultrasonic pulse to obtain the phase polarity of the third ultrasonic pulse, with transmission frequencies of the first ultrasonic pulse and the third ultrasonic pulse serving as a first frequency “f1”, and inverts the phase polarity of the second ultrasonic pulse to obtain the phase polarity of the fourth ultrasonic pulse, with transmission frequencies of the second ultrasonic pulse and the fourth ultrasonic pulse serving as a second frequency “f2” different from the first frequency. The combining unit 121a generates a first composite signal by adding the first reception signal to the third reception signal, and generates a second composite signal by adding the second reception signal to the fourth reception signal. The image generator 13 combines image data based on the first composite signal with image data based on the second composite signal, to generate image data.

Specifically, according to the second embodiment, transmission/reception with an ultrasonic pulse (first ultrasonic pulse) having the transmission waveform “f1+” is performed at the first time, and transmission/reception with an ultrasonic pulse (second ultrasonic pulse) having the transmission waveform “f2+” is performed at the second time as illustrated in the lower drawing of FIG. 8. In addition, in the second embodiment, transmission/reception with an ultrasonic pulse (third ultrasonic pulse) having the transmission waveform “f1−” is performed at the third time, and transmission/reception with an ultrasonic pulse (fourth ultrasonic pulse) having the transmission waveform “f2−” is performed at the fourth time, as illustrated in the lower drawing of FIG. 8.

Specifically, in the second embodiment, the second transmission performed in the conventional method is changed to the third transmission, and the third transmission performed in the conventional method is changed to the second transmission. In addition, in the second embodiment, addition processing “1+3” is performed to add a reception signal (first reception signal) obtained by the first transmission to a reception signal (third reception signal) obtained by the third transmission. This addition produces a composite signal (first composite signal) in which “tissue harmonic component: 2*Hf1” is extracted, as illustrated in the lower drawing of FIG. 8. In addition, in the second embodiment, addition processing “2+4” is performed to add a reception signal (second reception signal) obtained by the second transmission to a reception signal (fourth reception signal) obtained by the fourth transmission. This addition produces a composite signal (second composite signal) in which “tissue harmonic component: 2*Hf2” is extracted, as illustrated in the lower drawing of FIG. 8.

By the above restriction on the transmission order, in the addition “1+3” of the second embodiment, the transmission frequency of the fourth time serving as the previous transmission of the first time is the same as the transmission frequency of the second time serving as the previous transmission of the third time, that is, “f2”. In addition, by restriction on the above transmission/reception order, in the addition “2+4” of the second embodiment, the transmission frequency of the first time serving as the previous transmission of the second time is the same as the transmission frequency of the third time serving as the previous transmission of the fourth time, that is, “f1”.

By the above restriction on the transmission order, in the second embodiment, residual multiplex “f2−” of the fourth time on the adjacent scanning line is mixed into the reception signal of the first time, as illustrated in the lower drawing of FIG. 8. In addition, in the second embodiment, the residual multiplex “f1+” of the first time on the same scanning line is mixed into the reception signal of the second time, as illustrated in the lower drawing of FIG. 8. In addition, in the second embodiment, the residual multiplex “f2+” of the second time on the same scanning line is mixed into the reception signal of the third time, as illustrated in the lower drawing of FIG. 8. In addition, in the second embodiment, the residual multiplex “f1−” of the third time on the same scanning line is mixed into the reception signal of the fourth time, as illustrated in the lower drawing of FIG. 8.

However, in the second embodiment, addition “1+3” cancels the residual multiplex “f2−” and the residual multiplex “f2+”, and the residual multiplex of the composite signal (first composite signal) is “0”, as illustrated in the lower drawing of FIG. 8. In addition, in the second embodiment, addition “2+4” cancels the residual multiplex “f1+” and the residual multiplex “f1−”, and the residual multiplex of the composite signal (second composite signal) is “0”, as illustrated in the lower drawing of FIG. 8.

The above processing removes residual multiplex artifacts from B-mode image data generated from the composite signal including the extracted “tissue harmonic component: 2*Hf1”, and removes residual multiplex artifacts from B-mode image data generated from the composite signal including the extracted “tissue harmonic component: 2*Hf2”. As a result, composite image data (for example, arithmetically averaged image data) of these two B-mode image data serves as image data from which residual multiplex artifacts have been removed, and in which only “tissue harmonic component: 2*Hf1+2*Hf2” is imaged.

As described above, according to the second embodiment, the above restriction condition for the transmission order based on the transmission frequency is applied, to prevent occurrences of multiple residual artifacts, when image data having high image quality is obtained with a harmonic component of a broad band by applying PM. According to the second embodiment, by the above restriction condition for the transmission order, the fundamental wave components of residual multiplexes included in the two respective reception signals to be added have inverted phase polarities, and have no phase shift because they are caused by the transmission waveforms at the same transmission frequency. In this manner, according to the second embodiment, under the above restriction condition for the transmission order, it is possible to securely obtain image data having high image quality with a harmonic component of a broad band. The restriction condition for the transmission order is also applicable to THI using a difference sound component.

In addition, there may be the case where a modification obtained by combining the first embodiment with the second embodiment is performed. For example, in the modification, two ultrasonic transmissions/receptions performed based on PM with “transmission focus: F1” are performed at an optimum central frequency “f1” that is set based on the frequency attenuation characteristic. In addition, for example, in the modification, two ultrasonic transmissions/receptions performed based on PM with “transmission focus: F2” are performed at an optimum central frequency “f2” that is set based on the frequency attenuation characteristic.

In such a case, the controller 16 restricts the transmission order such that previous transmissions of two respective transmissions corresponding to the two reception signals in one set added by the combining unit 121a have an identical transmission focus position and an identical transmission frequency, to extract a harmonic component. For example, the transmitter/receiver 11 transmits the transmission waveform of f1 focused on F1 at the first time, and transmits a transmission waveform obtained by inverting the phase polarity of the transmission waveform at the third time. The transmitter/receiver 11 also transmits the transmission waveform of f2 focused on F2 at the second time, and transmits a transmission waveform obtained by inverting the phase polarity of the transmission waveform at the fourth time.

Specifically, in the lower drawing of FIG. 8, the pulse “f1+” transmitted at the first time serves as an ultrasonic pulse focused on “F1”, and the pulse “f2+” transmitted at the second time serves as an ultrasonic pulse focused on “F2”. In addition, in the lower drawing of FIG. 8, the pulse “f1−” transmitted at the third time serves as an ultrasonic pulse focused on “F1”, and the pulse “f2−” transmitted at the fourth time serves as an ultrasonic pulse focused on “F2”.

The combining unit 121a performs addition “1+3”, to generate a composite signal of “F1” from which residual multiplex components have been removed together with the fundamental wave components. The combining unit 121a also performs addition “2+4”, to generate a composite signal of “F2” from which residual multiplex components have been removed together with the fundamental wave components. In this manner, the modification enables removal of residual multiplex artifacts from image data of each transmission focus obtained with the central frequency suitable for each transmission focus.

### Third Embodiment

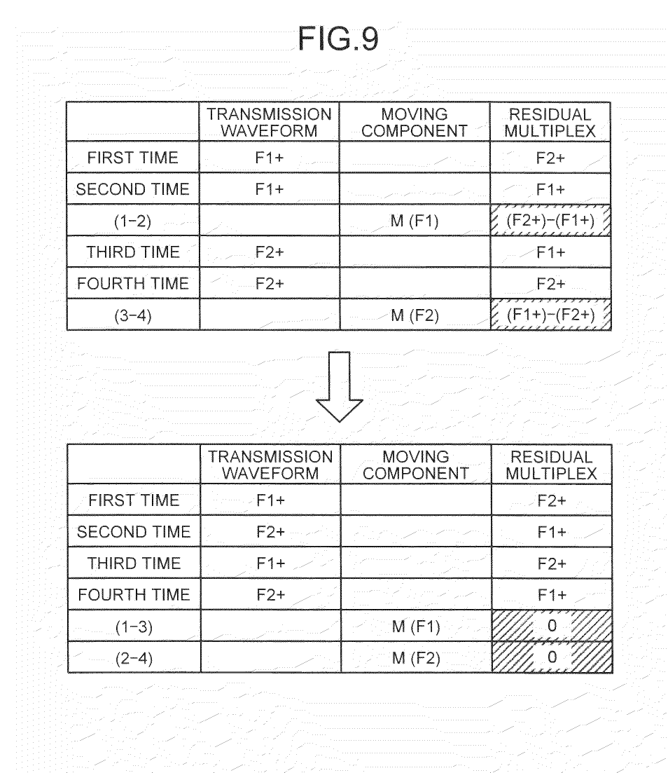

In a third embodiment, explained is the case where the transmission order is restricted when an ultrasonic transmission/reception set for extracting a signal component having a moving object as a reflection source is executed a plurality of times on the identical scanning line, with reference to FIG. 9. FIG. 9 is a diagram for explaining the third embodiment.

Specifically, according to the third embodiment, the transmitter/receiver 11 performs the following processing, in rate subtraction imaging (RSI) aimed at imaging a signal component (hereinafter referred to as a “moving component”) having a moving object as a reflection source. Specifically, the transmitter/receiver 11 according to the third embodiment executes the ultrasonic transmission/reception sets a plurality of times on the scanning line at different transmission focus positions, and generates a plurality of sets of reception signals, the ultrasonic transmission/reception sets including two ultrasonic transmissions/receptions serving as a unit and being executed on the identical scanning line with ultrasonic pulses of an identical phase polarity. In the above case, the transmission condition the transmitter/receiver 11 changes at each of the plurality of the sets is the transmission focus position.

In addition, in the third embodiment, the combining unit 121a performs subtraction processing on two reception signals in each of the plurality of the sets, and generates a plurality of composite signals on the scanning line.

For example, the transmitter/receiver 11 executes a first set including two ultrasonic transmissions/receptions with the transmission waveform “F1+” focused on the transmission focus “F1” on the same scanning line. In addition, for example, the transmitter/receiver 11 executes a second set including two ultrasonic transmissions/receptions with the transmission waveform “F2+” focused on the transmission focus “F2” on the same scanning line. The transmitter/receiver 11 then generates two reception signals of “F1+” and two reception signals of “F2+” on each scanning line.

The combining unit 121a subjects the two “F1+” reception signals to subtraction processing, to generate a composite signal that includes a remaining moving component “M (F1)” including a moving object (such as blood flow and ultrasonic contrast agent) that has moved from the scanning line as a reflection source between transmissions. The combining unit 121a also subjects the two “F2+” reception signals to subtraction processing, to generate a composite signal including an extracted moving component “M (F2)” including a moving object (such as a blood flow and an ultrasonic contrast) that has moved from the scanning line as a reflection source between transmissions.

RSI enables imaging of a moving object in a range including the transmission focus “F1” serving as the center, and a moving object in a range including the transmission focus “F2” serving as the center, using the composite signal of “M (F1)” and the composite signal of “M (F2)”.

In conventional art, as illustrated in the upper drawing of FIG. 9, the transmission waveform of an ultrasonic pulse that is transmitted at the first time is the transmission waveform “F1+”, the transmission waveform of an ultrasonic pulse that is transmitted at the second time is the transmission waveform “F1+”, the transmission waveform of an ultrasonic pulse that is transmitted at the third time is the transmission waveform “F2+”, and the transmission waveform of an ultrasonic pulse that is transmitted at the fourth time is the transmission waveform “F2+”.

However, in conventional art, the residual multiplex “F2+” of the fourth time on the adjacent scanning line is mixed into the reception signal obtained by the first transmission, as illustrated in the upper drawing of FIG. 9. In addition, in conventional art, the residual multiplex “F1+” of the first time on the same scanning line is mixed into the reception signal obtained by transmission of the second time, as illustrated in the upper drawing of FIG. 9. For this reason, in conventional art, “(F2+)−(F1+)” is left without being removed in the composite signal including “M (F1)”, as illustrated in the upper drawing of FIG. 9, by the subtraction “1−2”.

In addition, in conventional art, the residual multiplex “F1+” of the second time on the same scanning line is mixed into the reception signal obtained by the third transmission, as illustrated in the upper drawing of FIG. 9. In addition, in conventional art, the residual multiplex “F2+” of the third time on the same scanning line is mixed into the reception signal obtained by the fourth transmission, as illustrated in the upper drawing of FIG. 9. For this reason, in conventional art, “(F1+)−(F2+)” is left without being removed in the composite signal including “M (F2)”, as illustrated in the upper drawing of FIG. 9, by the subtraction “3−4”.

In contrast, the controller 16 according to the third embodiment controls the transmitter/receiver 11 such that previous transmissions of two respective transmissions corresponding to the two reception signals in one set subjected to subtraction processing by the combining unit 121a have an identical transmission focus position. The above subtraction processing is processing to extract a moving component.

In the example executed with the restriction on the transmission order, the transmitter/receiver 11 causes the ultrasonic probe 1 to execute ultrasonic transmission in the order of the first ultrasonic pulse, the second ultrasonic pulse, the third ultrasonic pulse, and the fourth ultrasonic pulse. The transmitter/receiver 11 inverts the phase polarity of the first ultrasonic pulse to obtain the phase polarity of the third ultrasonic pulse, with transmission focus positions of the first ultrasonic pulse and the third ultrasonic pulse serving as a first position “F1”, and invert the phase polarity of the second ultrasonic pulse to obtain the phase polarity of the fourth ultrasonic pulse, with transmission focus positions of the second ultrasonic pulse and the fourth ultrasonic pulse serving as a second position “F2” different from the first position. The combining unit 121a generates a first composite signal by performing subtraction processing on the first reception signal and the third reception signal, and generates a second composite signal by performing subtraction processing on the second reception signal and the fourth reception signal.

Specifically, in the third embodiment, transmission/reception with an ultrasonic pulse (first ultrasonic pulse) having the transmission waveform “F1+” is performed at the first time, and transmission/reception with an ultrasonic pulse (second ultrasonic pulse) having the transmission waveform “F2+” is performed at the second time as illustrated in the lower drawing of FIG. 9. In addition, in the third embodiment, transmission/reception with an ultrasonic pulse (third ultrasonic pulse) having the transmission waveform “F1+” is performed at the third time, and transmission/reception with an ultrasonic pulse (fourth ultrasonic pulse) having the transmission waveform “F2+” is performed at the fourth time, as illustrated in the lower drawing of FIG. 9.

Specifically, in the third embodiment, the second transmission performed in the conventional method is changed to the third transmission, and the third transmission performed in the conventional method is changed to the second transmission. In addition, in the third embodiment, subtraction processing “1−3” is performed on a reception signal (first reception signal) obtained by the first transmission and a reception signal (third reception signal) obtained by the third transmission. This subtraction processing produces a composite signal (first composite signal) including extracted “M (F1)”, as illustrated in the lower drawing of FIG. 9. In addition, in the third embodiment, subtraction processing “2−4” is performed between a reception signal (second reception signal) obtained by the second transmission and a reception signal (fourth reception signal) obtained by the fourth transmission. This subtraction produces a composite signal (second composite signal) including extracted “M (F2)”, as illustrated in the lower drawing of FIG. 9.

By the above restriction on the transmission order, in the subtraction “1−3” of the third embodiment, the transmission focus position of the fourth time serving as the previous transmission of the first time is the same as the transmission focus position of the second time serving as the previous transmission of the third time, that is, “F2”. In addition, by the above restriction on the transmission/reception order, in the subtraction “2−4” of the third embodiment, the transmission focus position of the first time serving as the previous transmission of the second time is the same as the transmission focus position of the third time serving as the previous transmission of the fourth time, that is, “F1”.

By the above restriction on the transmission order, in the third embodiment, residual multiplex “F2+” of the fourth time on the adjacent scanning line is mixed into the reception signal obtained by the first transmission, as illustrated in the lower drawing of FIG. 9. In addition, in the third embodiment, the residual multiplex “F1+” of the first time on the same scanning line is mixed into the reception signal obtained by the second transmission, as illustrated in the lower drawing of FIG. 9. In addition, in the third embodiment, the residual multiplex “F2+” of the second time on the same scanning line is mixed into the reception signal obtained by the third transmission, as illustrated in the lower drawing of FIG. 9. In addition, in the third embodiment, the residual multiplex “F1+” of the third time on the same scanning line is mixed into the reception signal obtained by the fourth transmission, as illustrated in the lower drawing of FIG. 9.

However, in the third embodiment, the subtraction “1−3” cancels the residual multiplex “F2+” and the residual multiplex “F2+”, and the residual multiplex of the composite signal is “0”, as illustrated in the lower drawing of FIG. 9. In addition, in the third embodiment, the subtraction “2−4” cancels the residual multiplex “F1+” and the residual multiplex “F1+”, and the residual multiplex of the composite signal is “0”, as illustrated in the lower drawing of FIG. 9.

As described above, according to the third embodiment, the above restriction condition for the transmission order based on the transmission focus position is applied, to prevent occurrences of multiple residual artifacts, when multi focusing is used together with RSI. According to the third embodiment, by the above restriction condition for the transmission order, the fundamental wave components of residual multiplexes included in the two respective reception signals to be subjected to subtraction processing have an identical phase polarity, and have no phase shift because they are originated from the transmission waveforms corresponding to the same transmission focus position. Therefore, the third embodiment can remove residual multiple artifacts under the above restriction condition for the transmission order even when multi focusing is used together with RSI.

### Fourth Embodiment

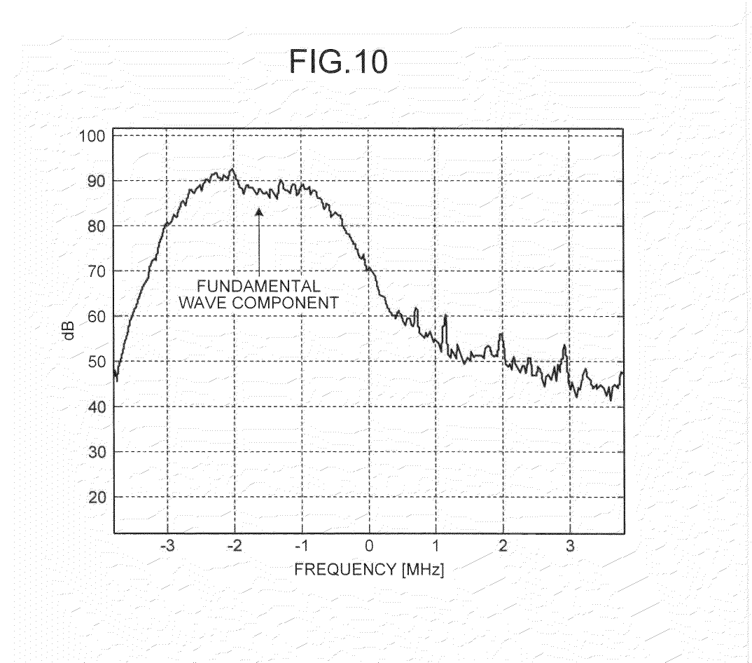

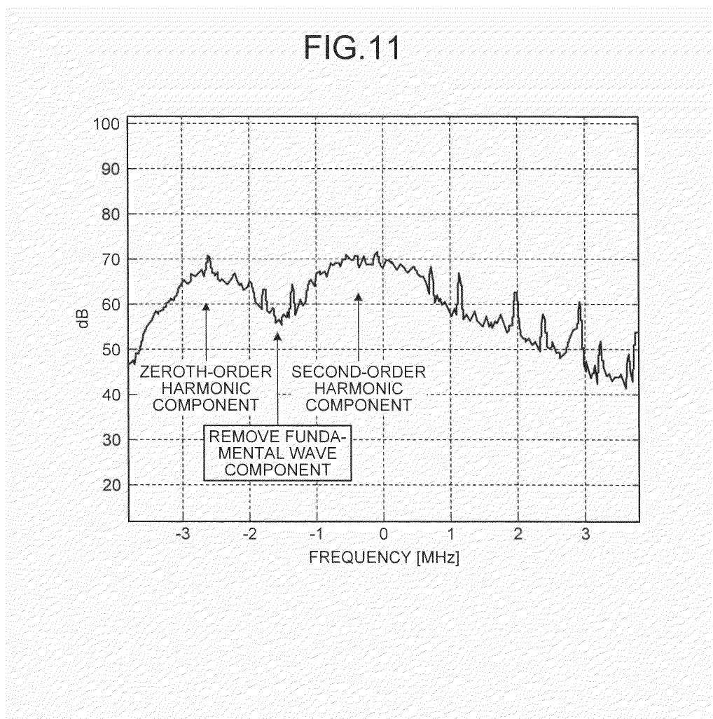

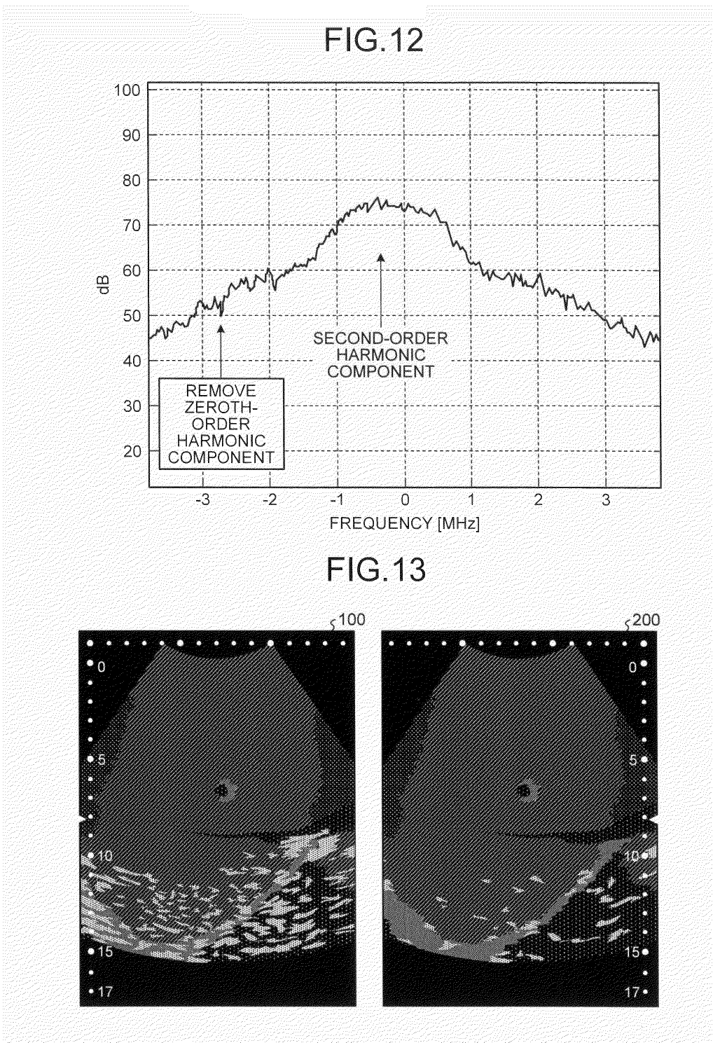

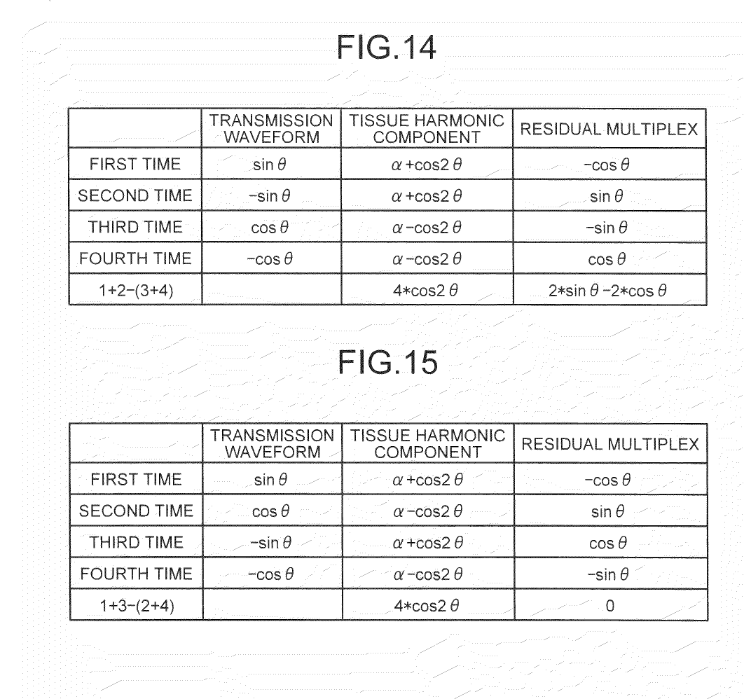

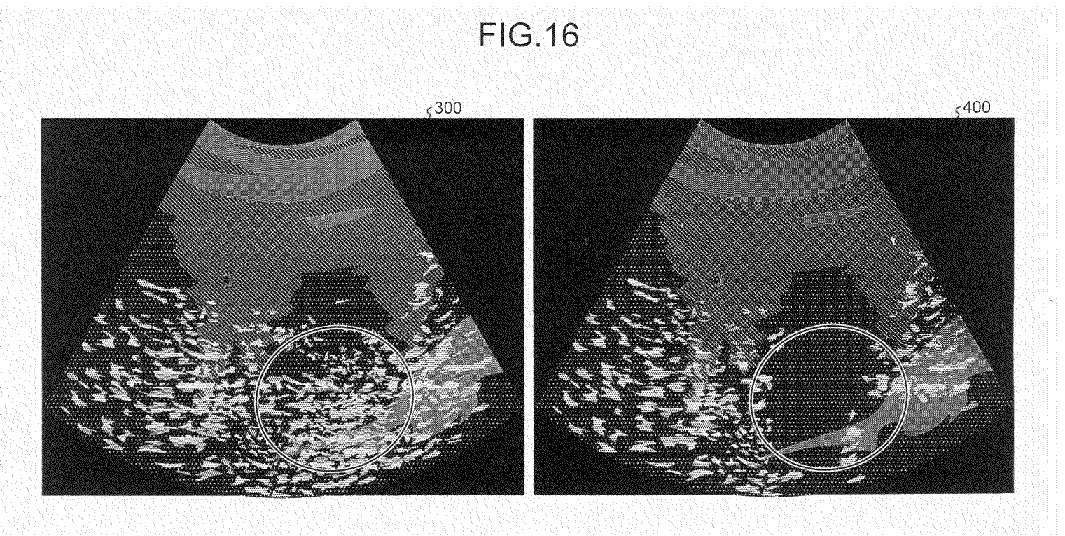

In a fourth embodiment, explained is the case of removing residual multiple artifacts in a scan sequence for removing a zeroth-order harmonic component as well as the fundamental wave components, by PM or THI using a difference sound component, with reference to numerical expressions and FIG. 10 to FIG. 16. FIG. 10 to FIG. 14 are diagrams for explaining a scan sequence for removing a zeroth-order harmonic component. FIG. 15 and FIG. 16 are diagrams for explaining a scan sequence for removing a zeroth-order harmonic component according to the fourth embodiment.

As explained in the first embodiment with reference to FIG. 3, harmonic components include a zeroth-order harmonic component, as well as harmonic components (such as second-order harmonic component) to be imaged. For example, when the transmission ultrasonic wave has a broad band, a zeroth-order harmonic component may overlap a second-order harmonic component. As another example, when the transmission ultrasonic wave has a broad band, a zeroth-order harmonic component may overlap a difference sound component.

In such a case, because the central frequency becomes lower due to influence caused by attenuation of the frequency dependence as the depth from the transmission position increases, a zeroth-order harmonic component comes to a level that cannot be ignored at the deep part. As a result, the deep-part resolution of the image deteriorates. The combining unit 121a is capable of removing a zeroth-order harmonic component by filtering. However, when the zeroth-order harmonic component is reduced by filtering or the like, the filtering also reduces the lower frequency range part of the second-order harmonic component, or the lower frequency range part of the difference sound component. This causes an image that is non-uniform in the depth direction due to insufficient penetration.

To avoid deterioration in the deep-part resolution caused by a zeroth-order harmonic component, the transmitter/receiver 11 and the signal processor 12 (combining part 121a) execute a scan sequence and combining processing explained below, under the control of the controller 16.

The transmitter/receiver 11 performs the following processing for the purpose of imaging harmonic components other than a zeroth-order harmonic component. Specifically, the transmitter/receiver 11 executes, on the identical scanning line, a first set of ultrasonic transmission/reception including two ultrasonic transmissions/receptions serving as a unit and being executed with inverted phase polarities of an ultrasonic pulse, and a second set of ultrasonic transmission/reception including two ultrasonic transmissions/receptions serving as a unit and being executed with inverted phase polarities of an ultrasonic pulse having a transmission waveform different from the transmission waveform of the ultrasonic pulse of the first set. The transmitter/receiver 11 also generates these two sets of reception signals. In the above case, the transmission condition the transmitter/receiver 11 changes at each of the sets is a transmission waveform. In addition, the combining unit 121a generates, on the scanning line, two composite signals including a composite signal obtained by adding two reception signals obtained by the first set of ultrasonic transmission/reception, and a composite signal obtained by adding two reception signals obtained by the second set of ultrasonic transmission/reception, performs subtraction processing on the two composite signals, and generates a composite signal on the scanning line. The image generator 13 generates ultrasonic image data using the composite signal. Specifically, the B-mode data generator 121b generates B-mode data from the composite signal of each scanning line, and the image generator 13 generates ultrasonic image data (B-mode image data) using the B-mode data.

For example, the transmitter/receiver 11 executes, on the same scanning line, the first set including first transmission with a first phase and second transmission with a second phase that is different from the first phase by 180°, and the second set including third transmission with a third phase that is different from the first phase by 90° and fourth transmission with a fourth phase that is different from the first phase by 270°. For example, the transmitter/receiver 11 performs the transmissions in the order of the first transmission, the second transmission, the third transmission, and the fourth transmission. The initial phase of the first transmission is denoted by “φ” hereinafter. In such a case, the initial phase of the second transmission is “φ+π”, the initial phase of the third transmission is “φ+π/2”, and the initial phase of the fourth transmission is “φ−π/2”.

Supposing that the ultrasonic pulse of the first transmission is denoted by “sin θ”, the ultrasonic pulse of the second transmission is “−sin θ”, the ultrasonic pulse of the third transmission is “cos θ”, and the ultrasonic pulse of the fourth transmission is “−cos θ”.

When the time is “t”, the envelope signal indicating the amplitude is “p (t)”, and the angular frequency serving as the central frequency is “ω”, the transmission signal (ultrasonic pulse) “S_{TX} (t)=p (t)cos (ωt+φ)” can be represented by the following Equation (1), using Euler's formula. The symbol “j” in Equation (1) represents the imaginary unit.

$$\begin{matrix}{S}_{TX} ( t ) = p ( t ) cos ( ω t + ϕ ) \\ = \frac{1}{2} p ( t ) \{ exp ( jω t + jϕ ) + exp ( - jω t - jϕ ) \}\end{matrix} \qquad ( 1 )$$

The second-order harmonic component “S_{H} (t)=S_{TX}² (t)=p² (t)cos² (ωt+φ)” serving as a second nonlinear component that is generated while “S_{TX} (t)” indicated in Equation (1) propagates in the tissue can be represented by the following Equation (2) using Euler's formula.

$$\begin{matrix}{S}_{H} ( t ) = {S}_{TX}^{2} ( t ) \\ = {p}^{2} ( t ) {cos}^{2} ( ω t + ϕ ) \\ = \frac{1}{4} {p}^{2} ( t ) \{ 2 + exp ( j 2 ω t + j2ϕ ) + exp ( - j 2 ω t - j2ϕ ) \} \\ = \frac{1}{2} {p}^{2} ( t ) + \frac{1}{4} {p}^{2} ( t ) \{ exp ( j 2 ω t + j2ϕ ) + exp ( - j 2 ω t - j2ϕ ) \}\end{matrix} \qquad ( 2 )$$

The signal obtained by adding the fundamental wave indicated in Equation (1) to the second nonlinear component indicated in Equation (2) reaches the target of the subject P and reflected therefrom. When “α” is a ratio of the “second-order nonlinear term” to “fundamental wave”, the signal obtained by adding the fundamental wave to the second nonlinear component is represented by the following Equation (3).

$$S ( t ) = \frac{1}{2} p ( t ) \{ exp ( jω t + jϕ ) + exp ( - j ω t - jϕ ) \} + \frac{α}{2} {p}^{2} ( t ) + \frac{α}{4} {p}^{2} ( t ) \{ exp ( j 2 ω t + j2ϕ ) + exp ( - j 2 ω t - j2ϕ ) \} \qquad ( 3 )$$

In accordance with an instruction from the controller 16, the transmitter/receiver 11 executes the first transmission with the first initial phase “φ”. The transmitter/receiver 11 subjects a reflected wave signal of the first transmission to amplification and reception delay addition processing or the like thereafter, to generate and output a reception signal “S1”. The reception signal “S1 (t)” having time “t” indicating the depth direction as a parameter can be represented by the following Equation (4). Equation (4) is based on the assumption that a harmonic wave generated by propagation through the transmission path is hardly attenuated through the reception path, and is identical to Equation (3).

$$S 1 ( t ) = \frac{1}{2} p ( t ) \{ exp ( jω t + jϕ ) + exp ( - j ω t - jϕ ) \} + \frac{α}{2} {p}^{2} ( t ) + \frac{α}{4} {p}^{2} ( t ) \{ exp ( j 2 ω t + j2ϕ ) + exp ( - j 2 ω t - j2ϕ ) \} \qquad ( 4 )$$

FIG. 10 illustrates a spectrum of the reception signal “S1”. In FIG. 10, the horizontal axis indicates the frequency (unit: MHz), and the vertical axis indicates the intensity (unit: dB) of the reception signal. As illustrated in FIG. 10, the frequency characteristic of the reception signal S1 has a spectrum in which the fundamental wave component is dominant.

Next, in accordance with an instruction from the controller 16, the transmitter/receiver 11 executes the second transmission with the second initial phase “φ+π”. The transmitter/receiver 11 subjects a reflected wave signal of the second transmission to amplification and reception delay addition processing thereafter, to generate and output a reception signal “S2”. The reception signal “S2 (t)” can be represented by the following Equation (5).

$$S 2 ( t ) = - \frac{1}{2} p ( t ) \{ exp ( jω t + jϕ ) + exp ( - j ω t - jϕ ) \} + \frac{α}{2} {p}^{2} ( t ) + \frac{α}{4} {p}^{2} ( t ) \{ exp ( j 2 ω t + j2ϕ ) + exp ( - j 2 ω t - j2ϕ ) \} \qquad ( 5 )$$

The combining unit 121a adds the reception signal “S1” and the reception signal “S2”. The added signal “S1 (t)+S2 (t)” can be represented by the following Equation (6).

$$S 1 ( t ) + S 2 ( t ) = α \cdot {p}^{2} ( t ) \frac{α}{2} {p}^{2} ( t ) \{ exp ( j 2 ω t + j2ϕ ) + exp ( - j 2 ω t - j2ϕ ) \} \qquad ( 6 )$$

In each of the right side of Equation (4) and the right side of Equation (5), the first term is a fundamental wave component, the second term is a zeroth-order harmonic component, and the third term is a second-order harmonic component. The zeroth-order harmonic component can be expressed simply with “α” and “p (t)”, as expressed in Equation (4) and Equation (5).

In addition, the first term of the right side of Equation (4) has a sign inversed to the sign of the first term of the right side of Equation (5). The second term of the right side of Equation (4) has the same sign as the sign of the second term of the right side of Equation (5). The third term of the right side of Equation (4) has the same sign as the sign of the third term of the right side of Equation (5). The added signal “S1 (t)+S2 (t)” is therefore a signal in which the fundamental wave components are cancelled and the zeroth-order harmonic component and the second-order harmonic component are doubled, as expressed in Equation (6).

FIG. 11 illustrates a spectrum of the added signal “S1+S2”. In FIG. 11, the horizontal axis indicates the frequency (unit: MHz), and the vertical axis indicates the intensity (unit: dB) of the reception signal. As illustrated in FIG. 11, the frequency characteristic of the added signal “S1+S2” includes a spectrum in which fundamental wave components are removed, and a zero-order component and a second-order harmonic component appear.

Next, in accordance with an instruction from the controller 16, the transmitter/receiver 11 executes the third transmission with the third initial phase “φ+π/2”. The transmitter/receiver 11 subjects a reflected wave signal of the third transmission to amplification and reception delay addition processing thereafter, to generate and output the reception signal “S3”. The reception signal “S3 (t)” can be represented by the following Equation (7).

$$S 3 ( t ) = \frac{j}{2} p ( t ) \{ exp ( jω t + jϕ ) - exp ( - j ω t - jϕ ) \} + \frac{α}{2} {p}^{2} ( t ) - \frac{α}{4} {p}^{2} ( t ) \{ exp ( j 2 ω t + j2ϕ ) + exp ( - j 2 ω t - j2ϕ ) \} \qquad ( 7 )$$

The combining unit 121a adds the added signal “S1+S2” to a signal obtained by multiplying the reception signal “S3” by −1. In other words, the combining unit 121a subtracts “S3” from “S1+S2”. The combining unit 121a then stores the signal “S1+S2−S3” in the memory.

Finally, in accordance with an instruction from the controller 16, the transmitter/receiver 11 executes the fourth transmission with the fourth initial phase “φ−π/2”. The transmitter/receiver 11 subjects a reflected wave signal of the fourth transmission to amplification and reception delay addition processing thereafter, to generate and output the reception signal “S4”. The reception signal “S4 (t)” can be represented by the following Equation (8).

$$S 4 ( t ) = - \frac{j}{2} p ( t ) \{ exp ( jω t + jϕ ) - exp ( - j ω t - jϕ ) \} + \frac{α}{2} {p}^{2} ( t ) - \frac{α}{4} {p}^{2} ( t ) \{ exp ( j 2 ω t + j2ϕ ) + exp ( - j 2 ω t - j2ϕ ) \} \qquad ( 8 )$$

The signal processor 12 (combining unit 121a) adds the signal “S1+S2−S3” to a signal obtained by multiplying the reception signal “S4” by −1. In other words, the combining unit 121a subtracts “S4” from “S1+S2−S3”. The combining unit 121a then obtains the signal “S1+S2−S3−S4”, that is, the signal “S1+S2−(S3+S4)” as the composite signal. The signal “S1 (t)+S2 (t)−S3 (t)−S4 (t)” using the time “t” indicating the depth direction can be represented by the following Equation (9).

$$\begin{matrix}S 1 ( t ) + S 2 ( t ) - S 3 ( t ) - S 4 ( t ) = α \cdot {p}^{2} ( t ) \{ exp ( j 2 ω t + j2ϕ ) + \\ exp ( - j 2 ω t - j2ϕ ) \} \\ = 2 α \cdot {p}^{2} ( t ) cos ( 2 ω t + 2 ϕ )\end{matrix} \qquad ( 9 )$$

In each of the right side of Equation (7) and the right side of Equation (8), the first term is the fundamental wave component, the second term is the zeroth-order harmonic component, and the third term is the second-order harmonic component. In addition, the first term of the right side of Equation (7) has a sign inversed to the sign of the first term of the right side of Equation (8). The second term of the right side of Equation (7) has the same sign as the sign of the second term of the right side of Equation (8). The third term of the right side of Equation (7) has the same sign as the sign of the third term of the right side of Equation (8). The signal “S3 (t)+S4 (t)” is a signal in which the fundamental wave components are cancelled and the zeroth-order harmonic component and the second-order harmonic component are doubled.

In addition, the zeroth-order harmonic component of “S1+S2” has the same sign as the sign of the zeroth-order harmonic component of “S3+S4”. By contrast, the second-order harmonic component of “S1+S2” has a sign inversed to the sign of the second-order harmonic component of “S3+S4”. When combining processing “S1+S2−(S3+S4)” is performed, the zero-order components are canceled as well as the fundamental wave components, and only the second-order harmonic components can be extracted, as shown in Equation (9). In other words, the signal “S1+S2−(S3+S4)” is a signal in which the second-order harmonic components included in the four reception signals are added. For example, the signal “S1+S2−(S3+S4)” is a signal in which the second-order harmonic component included in “S1” is amplified to have an intensity four times as large as the original intensity.

FIG. 12 illustrates a spectrum of the composite signal “S1+S2−(S3+S4)”. In FIG. 12, the horizontal axis indicates the frequency (unit: MHz), and the vertical axis indicates the intensity (unit: dB) of the reception signal. As illustrated in FIG. 12, the frequency characteristic of the composite signal “S1+S2−(S3+S4)” includes a spectrum in which the zeroth-order harmonic component is removed, and the second-order harmonic component is amplified.

The transmitter/receiver 11 executes the above four transmissions once in each of the scanning lines that form the scanning range for a frame (or for a volume). Next, the combining unit 121a generates, on each scanning line, a composite signal “S1+S2−(S3+S4)” obtained by combining the four reception signals (S1, S2, S3, S4) generated and output by the transmitter/receiver 11. The B-mode data generator 121b subjects the composite signal “S1+S2−(S3+S4)” of each scanning line output by the combining unit 121a to envelope wave detection and logarithm compression or the like thereafter, to generate B-mode data for a frame (or for a volume).

Thereafter, the image generator 13 generates B-mode image data from the B-mode data, and the monitor 2 displays B-mode image data under the control of the controller 16. This processing produces an image formed of a signal in which the fundamental wave components and the zeroth-order harmonic components are cancelled and only the second-order harmonic components are amplified.

Image data 100 illustrated in the left drawing of FIG. 13 is B-mode image data generated by executing, for example, the above first transmission and second transmission on each of the scanning lines. Image data 200 illustrated in the right drawing of FIG. 13 is B-mode image data generated by the above four transmissions. As illustrated in FIG. 13, in the image data 100, artifacts caused by zeroth-order harmonic components occur in the deep region, and the deep-part resolution deteriorates. By contrast, as illustrated in FIG. 13, in the image data 200, artifacts in the deep region disappear, and the deep-part resolution is improved.

Described above is the scan sequence and the combining processing method for removing zeroth-order harmonic components in THI using PM. The following describes a scan sequence and a combining processing method for removing zeroth-order harmonic components in THI using a difference sound component.

The transmitter/receiver 11 transmits a composite pulse four times or more on each scanning line. The composite pulse is obtained by mixing two frequencies, that is, a first frequency component (a first ultrasonic pulse at a first frequency) and a second frequency component (a second ultrasonic pulse at a second frequency). In this operation, the transmitter/receiver 11 executes “a first set of transmissions including a first transmission and a second transmission” and “a second set of transmissions including a third transmission and a fourth transmission” explained below on the same scanning line.

The above first transmission is transmission using a composite pulse having the first frequency component with the first phase, and the second frequency component with the second phase. The above second transmission is transmission using a composite pulse having the first frequency component with a phase that is different from the first phase by 180°, and the second frequency component with a phase that is different from the second phase by 180°.

The above third transmission is transmission using a composite pulse having the first frequency component with a phase that is different from the first phase by 90°, and the second frequency component with a phase that is different from the second phase by 270°. In addition, the above fourth transmission is transmission using a composite pulse having the first frequency component with a phase that is different from the first phase by 270°, and the second frequency component with a phase that is different from the second phase by 90°.

The following example describes the case where a set of ultrasonic transmissions using an ultrasonic pulse (composite pulse) obtained by mixing two frequencies (a single frequency of an angular frequency “ω₀” and a single frequency of an angular frequency “ω₁”) is performed on the same scanning line in the order of the first transmission, the second transmission, the third transmission, and the fourth transmission, and a reception beam (composite signal) is formed by addition and subtraction of four reception signals obtained by the set of transmissions. In the following explanation, the initial phase of the first transmission signal that is set at “ω₀” is denoted by “φ₀”, and the initial phase of the second transmission signal that is set at “ω₁” is denoted by “φ₁”. (φ₀, φ₁) is set under phase conditions for generating a difference sound component having the same polarity as that of the second-order harmonic component.

In such a case, in the first transmission, transmitted is an ultrasonic pulse obtained by mixing (ω₀, ω₁) with the initial phases (φ₀, φ₁). In the second transmission, transmitted is an ultrasonic pulse obtained by mixing (ω₀, ω₁) with the initial phases (φ₀+π, φ₁+π). In the third transmission, transmitted is an ultrasonic pulse obtained by mixing (ω₀, ω₁) with the initial phases (φ₀+π/2, φ₁−π/2). In the fourth transmission, transmitted is an ultrasonic pulse obtained by mixing (ω₀, ω₁) with the initial phases (φ₀−π/2, φ₁+π/2).

When the time is “t”, the envelope signal indicating the amplitude of the single frequency of the angular frequency “ω₀” serving as the central frequency is “p₀ (t)”, and the envelope signal indicating the amplitude of the single frequency of the angular frequency “ω₁” serving as the central frequency is “p₁ (t)”, the transmission signal “S_{TX} (t)” obtained by mixing and adding the two single frequency signals with the initial phases (φ₀, φ₁) can be represented by the following Equation (10).

S_{TX}(t)=p₀(t)cos(ω₀t+φ₀)+p₁(t)cos(ω₁t+φ₁) (10)

In Equation (10), supposing that “ω₀t+φ₀” is “θ₀” and “ω₁t+φ₁” is “θ₁”, the transmission waveform of the ultrasonic pulse of the first transmission is “p₀ (t) cos θ₀+p₁ (t) cos θ₁”. The transmission waveform of the ultrasonic pulse of the second transmission is “−(p₀ (t) cos θ₀+p₁ (t) cos θ₁)” because the initial phases thereof are (φ₀+π, φ₁+π). The transmission waveform of the ultrasonic pulse of the third transmission is “−p₀ (t) sin θ₀+p₁ (t) sin θ₁” because the initial phases thereof are (φ₀+π/2, φ₁−π/2). The transmission waveform of the ultrasonic pulse of the fourth transmission is “p₀ (t) sin θ₀+p₁ (t) sin θ₁” (=“−(−p₀ (t) sin θ₀+p₁ (t) sin θ₁)”) because the initial phases thereof are (φ₀−π/2, φ₁+π/2). Specifically, the ultrasonic pulse of the first transmission and the ultrasonic pulse of the second transmission have the same transmission waveform and inverted phase polarities. In addition, the ultrasonic pulse of the third transmission and the ultrasonic pulse of the fourth transmission have the same transmission waveform and inverted phase polarities.

The second-order harmonic component “S_{H} (t)=S_{TX}² (t)” serving as a second nonlinear component that occurs while the “S_{TX} (t)” expressed in Equation (10) propagates in the tissue can be represented by the following Equation (11), using Euler's formula. The symbol “j” in Equation (11) represents the imaginary unit.

$$\begin{matrix}{S}_{H} ( t ) = {S}_{TX}^{2} ( t ) \\ = {\{ {p}_{o} ( t ) cos ( {ω}_{0} t + {ϕ}_{0} ) + {p}_{1} ( t ) cos ( {ω}_{1} t + {ϕ}_{1} ) \}}^{2} \\ = \frac{1}{2} {p}_{0}^{2} ( t ) + \frac{1}{4} {p}_{0}^{2} ( t ) \{ exp ( j 2 {ω}_{0} t + {j2ϕ}_{0} ) + exp ( - j 2 {ω}_{0} t - {j2ϕ}_{0} ) \} + \\ \frac{1}{2} {p}_{1}^{2} ( t ) + \frac{1}{4} {p}_{1}^{2} ( t ) \{ exp ( j 2 {ω}_{1} t + {j2ϕ}_{1} ) + exp ( - j 2 {ω}_{1} t - {j2ϕ}_{1} ) \} + \\ \frac{1}{2} {p}_{0}^{2} ( t ) {p}_{1}^{2} ( t ) \{ exp ( j 2 {ω}_{0} t + {j2ϕ}_{0} ) + exp ( - j 2 {ω}_{0} t - {j2ϕ}_{0} ) \} \\ \{ exp ( j 2 {ω}_{0} t + {j2ϕ}_{0} ) + exp ( - j 2 {ω}_{0} t - {j2ϕ}_{0} ) \} \\ = \frac{1}{2} {p}_{0}^{2} ( t ) + \frac{1}{4} {p}_{0}^{2} ( t ) \{ exp ( j 2 {ω}_{0} t + {j2ϕ}_{0} ) + exp ( - j 2 {ω}_{0} t - {j2ϕ}_{0} ) \} + \\ \frac{1}{2} {p}_{1}^{2} ( t ) + \frac{1}{4} {p}_{1}^{2} ( t ) \{ exp ( j 2 {ω}_{1} t + {j2ϕ}_{1} ) + exp ( - j 2 {ω}_{1} t - {j2ϕ}_{1} ) \} + \\ \frac{1}{2} {p}_{0}^{2} ( t ) {p}_{1}^{2} ( t ) \{ exp ( j 2 ( {ω}_{1} + {ω}_{0} ) t + j2 ( {ϕ}_{1} + {ϕ}_{0} ) ) + \\ exp ( - j ( {ω}_{1} + {ω}_{0} ) 2 t - j2 ( {ϕ}_{1} + {ϕ}_{0} ) ) \} + \\ \frac{1}{2} {p}_{0}^{2} ( t ) {p}_{1}^{2} ( t ) \{ exp ( j 2 ( {ω}_{1} - {ω}_{0} ) t + j2 ( {ϕ}_{1} - {ϕ}_{0} ) ) + \\ exp ( - j ( {ω}_{1} - {ω}_{0} ) 2 t - j2 ( {ϕ}_{1} - {ϕ}_{0} ) ) \}\end{matrix} \qquad ( 11 )$$

In the right side of Equation (11), the first term is a zeroth-order harmonic component of “ω₀”, and the second term is a second-order harmonic component of “ω₀”. In the right side of Equation (11), the third term is a zeroth-order harmonic component of “ω₁”, and the fourth term is a second-order harmonic component of “ω₁”. In addition, in the right side of Equation (11), the fifth term is an addition sound component (sum frequency component) of “ω₀” and “ω₁”, and the sixth term is a difference sound component (difference frequency component) of “ω₀” and “ω₁”.

The signal obtained by adding the fundamental wave expressed in Equation (10) to the second nonlinear component expressed in Equation (11) reaches the target of the subject P and is reflected therefrom. When “α” is a ratio of the “second-order nonlinear term” to the “fundamental wave”, the signal obtained by adding the fundamental wave and the second nonlinear component is represented by the following Equation (12).

$$S ( t ) = \frac{1}{2} {p}_{0} ( t ) \{ exp ( {jω}_{0} t + {jϕ}_{0} ) + exp ( - {jω}_{0} t - {jϕ}_{0} ) \} + \frac{1}{2} {p}_{1} ( t ) \{ exp ( {jω}_{1} t + {jϕ}_{1} ) + exp ( - {jω}_{1} t - {jϕ}_{1} ) \} + \frac{α}{2} {p}_{0}^{2} ( t ) + \frac{α}{4} {p}_{0}^{2} ( t ) \{ exp ( j 2 {ω}_{0} t + {j2ϕ}_{0} ) + exp ( - j 2 {ω}_{0} t - {j2ϕ}_{0} ) \} + \frac{α}{2} {p}_{1}^{2} ( t ) + \frac{α}{4} {p}_{1}^{2} ( t ) \{ exp ( j 2 {ω}_{1} t + {j2ϕ}_{1} ) + exp ( - j 2 {ω}_{1} t - {j2ϕ}_{1} ) \} + \frac{α}{2} {p}_{0}^{2} ( t ) {p}_{1}^{2} ( t ) \{ exp ( j ( {ω}_{1} + {ω}_{0} ) t + j ( {ϕ}_{1} + {ϕ}_{0} ) ) + exp ( - j ( {ω}_{1} + {ω}_{0} ) t - j ( {ϕ}_{1} + {ϕ}_{0} ) ) \} + \frac{α}{2} {p}_{0}^{2} ( t ) {p}_{1}^{2} ( t ) \{ exp ( j ( {ω}_{1} - {ω}_{0} ) t + j ( {ϕ}_{1} - {ϕ}_{0} ) ) + exp ( - j ( {ω}_{1} - {ω}_{0} ) t - j ( {ϕ}_{1} - {ϕ}_{0} ) ) \} \qquad ( 12 )$$

In accordance with an instruction from the controller 16, the transmitter/receiver 11 executes the first transmission in which (ω₀, ω₁) have the initial phases (φ₀, φ₁). The transmitter/receiver 11 subjects a reflected wave signal of the first transmission to amplification and reception delay addition processing or the like thereafter, to generate and output a reception signal “S1”. The reception signal “S1 (t)” having time “t” indicating the depth direction as a parameter can be represented by the following Equation (13).

$$S 1 ( t ) = \frac{1}{2} {p}_{0} ( t ) \{ exp ( {jω}_{0} t + {jϕ}_{0} ) + exp ( - {jω}_{0} t - {jϕ}_{0} ) \} + \frac{1}{2} {p}_{1} ( t ) \{ exp ( {jω}_{1} t + {jϕ}_{1} ) + exp ( - {jω}_{1} t - {jϕ}_{1} ) \} + \frac{α}{2} {p}_{0}^{2} ( t ) + \frac{α}{4} {p}_{0}^{2} ( t ) \{ exp ( j 2 {ω}_{0} t + {j2ϕ}_{0} ) + exp ( - j 2 {ω}_{0} t - {j2ϕ}_{0} ) \} + \frac{α}{2} {p}_{1}^{2} ( t ) + \frac{α}{4} {p}_{1}^{2} ( t ) \{ exp ( j 2 {ω}_{1} t + {j2ϕ}_{1} ) + exp ( - j 2 {ω}_{1} t - {j2ϕ}_{1} ) \} + \frac{α}{2} {p}_{0}^{2} ( t ) {p}_{1}^{2} ( t ) [ exp \{ j ( {ω}_{1} + {ω}_{0} ) t + j ( {ϕ}_{1} + {ϕ}_{0} ) \} + exp \{ - j ( {ω}_{1} + {ω}_{0} ) t - j ( {ϕ}_{1} + {ϕ}_{0} ) \} ] + \frac{α}{2} {p}_{0}^{2} ( t ) {p}_{1}^{2} ( t ) [ exp \{ j ( {ω}_{1} - {ω}_{0} ) t + j ( {ϕ}_{1} - {ϕ}_{0} ) \} + exp \{ - j ( {ω}_{1} - {ω}_{0} ) t - j ( {ϕ}_{1} - {ϕ}_{0} ) \} ] \qquad ( 13 )$$

Thereafter, in accordance with an instruction from the controller 16, the transmitter/receiver 11 executes the second transmission in which (ω₀, ω₁) have the initial phases (φ₀+π, φ₁+π), to generate and output a reception signal “S2”. The transmitter/receiver 11 also executes the third transmission in which (ω₀, ω₁) have the initial phases (φ₀+π/2, φ₁−π/2), to generate and output a reception signal “S3”. The transmitter/receiver 11 also executes the fourth transmission in which (ω₀, ω₁) have the initial phases (φ₀−π/2, φ₁+π/2), to generate and output a reception signal “S4”.

The reception signal “S2 (t)” can be represented by the following Equation (14), the reception signal “S3 (t)” can be represented by the following Equation (15), and the reception signal “S4 (t)” can be represented by the following Equation (16).

$$\begin{matrix}S 2 ( t ) = - \frac{1}{2} {p}_{0} ( t ) \{ exp ( {jω}_{0} t + {jϕ}_{0} ) + exp ( - {jω}_{0} t - {jϕ}_{0} ) \} - \frac{1}{2} {p}_{1} ( t ) \{ exp ( {jω}_{1} t + {jϕ}_{1} ) + exp ( - {jω}_{1} t - {jϕ}_{1} ) \} + \frac{α}{2} {p}_{0}^{2} ( t ) + \frac{α}{4} {p}_{0}^{2} ( t ) \{ exp ( j 2 {ω}_{0} t + {j2ϕ}_{0} ) + exp ( - j 2 {ω}_{0} t - {j2ϕ}_{0} ) \} + \frac{α}{2} {p}_{1}^{2} ( t ) + \frac{α}{4} {p}_{1}^{2} ( t ) \{ exp ( j 2 {ω}_{1} t + {j2ϕ}_{1} ) + exp ( - j 2 {ω}_{1} t - {j2ϕ}_{1} ) \} + \frac{α}{2} {p}_{0}^{2} ( t ) {p}_{1}^{2} ( t ) [ exp \{ j ( {ω}_{1} + {ω}_{0} ) t + j ( {ϕ}_{1} + {ϕ}_{0} ) \} + exp \{ - j ( {ω}_{1} + {ω}_{0} ) t - j ( {ϕ}_{1} + {ϕ}_{0} ) \} ] + \frac{α}{2} {p}_{0}^{2} ( t ) {p}_{1}^{2} ( t ) [ exp \{ j ( {ω}_{1} - {ω}_{0} ) t + j ( {ϕ}_{1} - {ϕ}_{0} ) \} + exp \{ - j ( {ω}_{1} - {ω}_{0} ) t - j ( {ϕ}_{1} - {ϕ}_{0} ) \} ] & ( 14 ) \\ S 3 ( t ) = \frac{j}{2} {p}_{0} ( t ) \{ exp ( {jω}_{0} t + {jϕ}_{0} ) - exp ( - {jω}_{0} t - {jϕ}_{0} ) \} - \frac{j}{2} {p}_{1} ( t ) \{ exp ( {jω}_{1} t + {jϕ}_{1} ) - exp ( - {jω}_{1} t - {jϕ}_{1} ) \} + \frac{α}{2} {p}_{0}^{2} ( t ) - \frac{α}{4} {p}_{0}^{2} ( t ) \{ exp ( j 2 {ω}_{0} t + {j2ϕ}_{0} ) + exp ( - j 2 {ω}_{0} t - {j2ϕ}_{0} ) \} + \frac{α}{2} {p}_{1}^{2} ( t ) - \frac{α}{4} {p}_{1}^{2} ( t ) \{ exp ( j 2 {ω}_{1} t + {j2ϕ}_{1} ) + exp ( - j 2 {ω}_{1} t - {j2ϕ}_{1} ) \} + \frac{α}{2} {p}_{0}^{2} ( t ) {p}_{1}^{2} ( t ) [ exp \{ j ( {ω}_{1} + {ω}_{0} ) t + j ( {ϕ}_{1} + {ϕ}_{0} ) \} + exp \{ - j ( {ω}_{1} + {ω}_{0} ) t - j ( {ϕ}_{1} + {ϕ}_{0} ) \} ] - \frac{α}{2} {p}_{0}^{2} ( t ) {p}_{1}^{2} ( t ) [ exp \{ j ( {ω}_{1} - {ω}_{0} ) t + j ( {ϕ}_{1} - {ϕ}_{0} ) \} + exp \{ - j ( {ω}_{1} - {ω}_{0} ) t - j ( {ϕ}_{1} - {ϕ}_{0} ) \} ] & ( 15 ) \\ S 4 ( t ) = - \frac{j}{2} {p}_{0} ( t ) \{ exp ( {jω}_{0} t + {jϕ}_{0} ) - exp ( - {jω}_{0} t - {jϕ}_{0} ) \} + \frac{j}{2} {p}_{1} ( t ) \{ exp ( {jω}_{1} t + {jϕ}_{1} ) - exp ( - {jω}_{1} t - {jϕ}_{1} ) \} + \frac{α}{2} {p}_{0}^{2} ( t ) - \frac{α}{4} {p}_{0}^{2} ( t ) \{ exp ( j 2 {ω}_{0} t + {j2ϕ}_{0} ) + exp ( - j 2 {ω}_{0} t - {j2ϕ}_{0} ) \} + \frac{α}{2} {p}_{1}^{2} ( t ) - \frac{α}{4} {p}_{1}^{2} ( t ) \{ exp ( j 2 {ω}_{1} t + {j2ϕ}_{1} ) + exp ( - j 2 {ω}_{1} t - {j2ϕ}_{1} ) \} + \frac{α}{2} {p}_{0}^{2} ( t ) {p}_{1}^{2} ( t ) [ exp \{ j ( {ω}_{1} + {ω}_{0} ) t + j ( {ϕ}_{1} + {ϕ}_{0} ) \} + exp \{ - j ( {ω}_{1} + {ω}_{0} ) t - j ( {ϕ}_{1} + {ϕ}_{0} ) \} ] - \frac{α}{2} {p}_{0}^{2} ( t ) {p}_{1}^{2} ( t ) [ exp \{ j ( {ω}_{1} - {ω}_{0} ) t + j ( {ϕ}_{1} - {ϕ}_{0} ) \} + exp \{ - j ( {ω}_{1} - {ω}_{0} ) t - j ( {ϕ}_{1} - {ϕ}_{0} ) \} ] & ( 16 )\end{matrix}$$

The combining unit 121a performs arithmetic processing of “S1+S2−S3−S4”, to generate a composite signal. Specifically, the combining unit 121a performs arithmetic processing of “S1+S2−(S3+S4)”. The composite signal “S1 (t)+S2 (t)−S3 (t)−S4 (t)” having time “t” indicating the depth direction as a parameter can be represented by the following Equation (17).

$$S 1 ( t ) + S 2 ( t ) - S 3 ( t ) - S 4 ( t ) = α {p}_{0}^{2} ( t ) \{ exp ( {j2ω}_{0} t + {j2ϕ}_{0} ) + exp ( - {j2ω}_{0} t - {j2ϕ}_{0} ) \} + α {p}_{0}^{2} ( t ) \{ exp ( {j2ω}_{0} t + {j2ϕ}_{0} ) + exp ( - {j2ω}_{0} t - {j2ϕ}_{0} ) \} + 2 α {p}_{0}^{2} ( t ) {p}_{1}^{2} ( t ) [ exp \{ j ( {ω}_{1} - {ω}_{0} ) t + j ( {ϕ}_{1} - {ϕ}_{0} ) \} + exp \{ - j ( {ω}_{1} - {ω}_{0} ) t - j ( {ϕ}_{1} - {ϕ}_{0} ) \} ] = 2 α {p}_{0}^{2} ( t ) cos ( 2 {ω}_{0} t + {ϕ}_{0} ) + 2 α {p}_{1}^{2} ( t ) cos ( 2 {ω}_{1} t + {ϕ}_{1} ) + 4 α {p}_{0}^{2} {p}_{1}^{2} cos \{ ( {ω}_{1} - {ω}_{0} ) t + ( {ϕ}_{1} - {ϕ}_{0} ) \} \qquad ( 17 )$$

In the composite signal represented by Equation (17), the fundamental wave components, the zeroth-order harmonic components, and the addition sound component (sum frequency component) of “ω₀” and “ω₁” are removed. In the composite signal represented by Equation (17), the second-order harmonic component (first term) of “ω₀”, the second-order harmonic component (second term) of “ω₁”, and the difference sound component (third term) of “ω₀” and “ω₁” are amplified and remain. In the case of “ω₀<ω₁”, the second-order harmonic component of “ω₁” may be set to fall out of the band that can be received by the ultrasonic probe 1. As another example, the second-order harmonic component of “ω₁” may be removed by filtering.

The transmitter/receiver 11 executes the above four transmissions once in each of the scanning lines that form the scanning range for a frame (or for a volume). Next, the combining unit 121a generates, on each scanning line, a composite signal “S1+S2−S3−S4” obtained by combining the four reception signals (S1, S2, S3, S4) generated and output by the transmitter/receiver 11. The B-mode data generator 121b subjects the composite signal “S1+S2−S3−S4” of each scanning line output by the combining unit 121a to envelope wave detection and logarithm compression or the like thereafter, to generate B-mode data for a frame (or for a volume). Thereafter, the image generator 13 generates B-mode image data from the B-mode data, and the monitor 2 displays B-mode image data under the control of the controller 16.

This processing enables generation and display of B-mode image data formed of a signal in which the fundamental wave components and the zeroth-order harmonic components are cancelled and the second-order harmonic components and the difference sound component (difference frequency component) are amplified.

In the above two types of “scan sequence and combining method” explained above, zeroth-order harmonic components can be removed even when the transmission order is changed to a desired order, as long as the relation between the phases and the relation between the addition and the subtraction are maintained. In addition, in the above two types of “scan sequence and combining method” explained above, zeroth-order harmonic components can be removed also by executing the four transmissions a plurality of times on each scanning line. However, the above two types of “scan sequence and combining method” explained above may fail to cancel multiplex of the fundamental wave components and residual multiplex artifacts might occur, due to a difference in transmission waveforms transmitted for removing the zeroth-order harmonic components. FIG. 14 illustrates that a scan sequence for removing a zeroth-order harmonic component in THI using PM is performed in which the first transmission is performed at the first time, the second transmission is performed at the second time, the third transmission is performed at the third time, and the fourth transmission is performed at the fourth time.

FIG. 14 also illustrates “a” as a zeroth-order harmonic component included in the reception signal. In such a case, the tissue harmonic component (zeroth-order harmonic component+second-order harmonic component) included in the reception signal originated from “transmission waveform: sin θ” of the first time is “α+cos 2θ” as illustrated in FIG. 14. The tissue harmonic component included in the reception signal originated from “transmission waveform: −sin θ” of the second time is “α+cos 2θ” as illustrated in FIG. 14.

In addition, the tissue harmonic component included in the reception signal originated from “transmission waveform: cos θ” of the third time is “α−cos 2θ” as illustrated in FIG. 14. The tissue harmonic component included in the reception signal originated from “transmission waveform: −cos θ” of the fourth time is “α−cos 2θ” as illustrated in FIG. 14. The combining unit 121a performs the addition “1+2” to remove the fundamental wave components from the reception signal obtained by the first transmission and the reception signal obtained by the second transmission, and performs the addition “3+4” to remove the fundamental wave components from the reception signal obtained by the third transmission and the reception signal obtained by the fourth transmission. The combining unit 121a performs the subtraction “(1+2)−(3+4)” thereafter to generate a composite signal in which “α” is removed and the second-order harmonic component is amplified to “4*cos 2θ”.

However, as illustrated in FIG. 14, the residual multiplex “−cos θ” of the fourth time on the adjacent scanning line is mixed into the reception signal obtained by the first transmission. In addition, as illustrated in FIG. 14, the residual multiplex “sin θ” of the first time on the same scanning line is mixed into the reception signal obtained by the second transmission. The residual multiplex “−sin θ” of the second time on the same scanning line is mixed into the reception signal obtained by the third transmission, as illustrated in FIG. 14. The residual multiplex “cos θ” of the third time on the same scanning line is mixed into the reception signal obtained by the fourth transmission, as illustrated in FIG. 14. In conventional art, therefore, “2*sin θ−2*cos θ” remains in the composite signal by the addition and subtraction processing “(1+2)−(3+4)”, as illustrated in FIG. 14, and residual multiplex artifacts occur.

Such residual multiplex artifacts occur also in the case of performing THI using a difference sound component, in which the first transmission is performed at the first time, the second transmission is performed at the second time, the third transmission is performed at the third time, and the fourth transmission performed at the fourth time in the scan sequence for removing zeroth-order harmonic components.

To remove such residual multiplex artifacts, the controller 16 according to the fourth embodiment controls the transmitter/receiver 11 such that previous transmissions of two respective transmissions corresponding to the two reception signals in one set added by the combining unit 121a have an identical transmission waveform. The above addition serves as processing for extracting harmonic components other than zeroth-order harmonic components.

In an example of processing executed with the restriction on the transmission order, the transmitter/receiver 11 causes the ultrasonic probe 1 to execute ultrasonic transmission in the order of the first ultrasonic pulse, the second ultrasonic pulse, the third ultrasonic pulse, and the fourth ultrasonic pulse. In the operation, the transmitter/receiver 11 inverts the phase polarity of the first ultrasonic pulse to obtain the phase polarity of the third ultrasonic pulse, with transmission waveforms of the first ultrasonic pulse and the third ultrasonic pulse serving as a first waveform (such as sin θ), and inverts the phase polarity of the second ultrasonic pulse to obtain the phase polarity of the fourth ultrasonic pulse, with transmission waveforms of the second ultrasonic pulse and the fourth ultrasonic pulse serving as a second waveform (such as cos θ) different from the first waveform. The combining unit 121a generates a first composite signal by adding the first reception signal to the third reception signal, generates a second composite signal by adding the second reception signal to the fourth reception signal, and generates a composite signal by performing subtraction processing on the first composite signal and the second composite signal. The image generator 13 generates image data based on the composite signal.

Specifically, in the fourth embodiment, in a scan sequence for removing zeroth-order harmonic components in THI using PM, transmission/reception is performed at the first time using an ultrasonic pulse (first ultrasonic pulse) with the transmission waveform “sin θ”, and transmission/reception is performed at the second time using an ultrasonic pulse (second ultrasonic pulse) with the transmission waveform “cos θ”, as illustrated in FIG. 15. In addition, in the fourth embodiment, in a scan sequence for removing zeroth-order harmonic components in THI using PM, transmission/reception is performed at the third time using an ultrasonic pulse (third ultrasonic pulse) with the transmission waveform “−sin θ”, and transmission/reception is performed at the fourth time using an ultrasonic pulse (fourth ultrasonic pulse) with the transmission waveform “−cos θ”, as illustrated in FIG. 15.

Specifically, in the fourth embodiment, the second transmission illustrated in FIG. 14 is changed to the third transmission, and the third transmission illustrated in FIG. 14 is changed to the second transmission. In addition, in the fourth embodiment, addition processing “1+3” is performed to add a reception signal (first reception signal) obtained by the first transmission to a reception signal (third reception signal) obtained by the third transmission. This addition produces a composite signal (first composite signal) in which “tissue harmonic component: 2α+2*cos 2θ” is extracted. In addition, in the fourth embodiment, addition processing “2+4” is performed to add a reception signal (second reception signal) obtained by the second transmission to a reception signal (fourth reception signal) obtained by the fourth transmission. This addition produces a composite signal (second composite signal) in which “tissue harmonic component: 2α−2*cos 2θ” is extracted. Subtraction processing “1+3−(2+4)” is performed on the two composite signals thereafter, to obtain a composite signal in which “tissue harmonic component: 4*cos 2θ” is extracted.

By the above restriction on the transmission order, in the addition “1+3” of the fourth embodiment, the transmission waveform of the fourth time serving as the previous transmission of the first time is the same as the transmission waveform of the second time serving as the previous transmission of the third time, that is, “cos θ”, although their phase polarities are inverted from each other. In addition, by the above restriction on the transmission order, in the addition “2+4” of the fourth embodiment, the transmission waveform of the first time serving as the previous transmission of the second time is the same as the transmission waveform of the third time serving as the previous transmission of the fourth time, that is, “sin θ”, although their phase polarities are inverted from each other.

By the above restriction on the transmission order, in the fourth embodiment, residual multiplex “−cos θ” of the fourth time on the adjacent scanning line is mixed into the reception signal obtained by the first transmission, as illustrated in FIG. 15. In addition, in the fourth embodiment, the residual multiplex “sin θ” of the first time on the same scanning line is mixed into the reception signal obtained by the second transmission, as illustrated in FIG. 15. In addition, in the fourth embodiment, the residual multiplex “cos θ” of the second time on the same scanning line is mixed into the reception signal obtained by the third transmission, as illustrated in FIG. 15. In addition, in the fourth embodiment, the residual multiplex “−sin θ” of the third time on the same scanning line is mixed into the reception signal obtained by the fourth transmission, as illustrated in FIG. 15.

However, in the fourth embodiment, the addition and subtraction processing “1+3−(2+4)” cancels the residual multiplex components, whereby the residual multiplex is reduced to “0”, and a composite signal is obtained in which “tissue harmonic component: 4*cos 2θ” is extracted, as illustrated in FIG. 15. As a result, the fourth embodiment can remove residual multiplex artifacts from image data obtained by THI using PM, together with artifacts caused by zeroth-order harmonic components

In the fourth embodiment, in the scan sequence for removing zeroth-order harmonic components in THI using a difference sound component, the transmission of the second time is changed to the transmission of the third time, and the transmission of the third time illustrated in FIG. 14 is changed to the transmission of the second time, by the restriction on the transmission order based on the above transmission waveform. Specifically, in the scan sequence for removing zeroth-order harmonic components in THI using a difference sound component according to the fourth embodiment, the first transmission is performed with the transmission waveform of “p₀ (t) cos θ₀+p₁ (t)cos θ₁”, and the second transmission is performed with the transmission waveform of “−p₀ (t)sin θ₀+p₁ (t)sin θ₁”. In addition, in the scan sequence, the third transmission is performed with the transmission waveform of “−(p₀ (t)cos θ₀+p₁ (t)cos θ₁)”, and the fourth transmission is performed with the transmission waveform of “−(−p₀ (t)sin θ₀+p₁ (t)sin θ₁)”, by the above restriction on the transmission order.

Subsequently, the combining unit 121a performs combining processing by the addition and subtraction processing “1+3−(2+4)”. The addition and subtraction processing “l+3−(2+4)” cancels the multiplex residual components, reduces the residual multiplex to “0”, and produces a composite signal in which the “difference sound component and second-order harmonic component” are amplified and extracted. As a result, the fourth embodiment can remove residual multiplex artifacts from image data obtained by THI using a difference sound component, together with artifacts caused by zeroth-order harmonic components.

Image data 300 illustrated in the left drawing of FIG. 16 is B-mode image data that is generated and displayed by executing the scan sequence for removing zeroth-order harmonic components in THI using a difference sound component in the transmission order before changing. Image data 400 illustrated in the right drawing of FIG. 16 is B-mode image data that is generated and displayed by executing the scan sequence for removing zeroth-order harmonic components in THI using a difference sound component in the transmission order changed under the above restriction conditions. As illustrated in FIG. 16, in the image data 300, residual multiplex artifacts occur in the deep region. By contrast, residual multiplex artifacts disappear from the image data 400, as illustrated in FIG. 16.

As described above, the fourth embodiment can remove residual multiplex artifacts from image data obtained by THI, together with artifacts caused by zeroth-order harmonic components, by the restriction on the transmission order based on the transmission waveform.

The scan sequence for removing zeroth-order harmonic components in the THI may be performed in which the transmission frequency of the first set and the transmission frequency of the second set are changed, to broaden the band of the harmonic components. The restriction condition for the transmission order in such a case is a restriction condition in which previous transmissions of two respective transmissions corresponding to the two reception signals in one set added by the combining unit 121a have an identical transmission frequency and an identical transmission waveform. Under the restriction condition, it is possible to remove residual multiplex artifacts, even when the transmission frequency of the first set and the transmission frequency of the second set are changed, to broaden the band of the harmonic components.

### Fifth Embodiment

In the above first to fourth embodiments, restricting the transmission order for removing residual multiplex artifacts broadens the interval between transmissions for obtaining two reception signals to be subjected to addition or subtraction (subtraction processing) to extract a signal component for imaging, in comparison with the interval in the scan sequence before change.

In such a case, difference in time due to the broadened transmission interval may cause motion artifacts caused by body motion. Specifically, the possibility that motion artifacts occur may increase, although multiplex artifacts can be removed by the transmission order explained in the first to the fourth embodiments.

However, residual multiplex artifacts are hard to occur when the ultrasonic image data has a small display depth, or when transmission pulses have short pulse intervals. Specifically, residual multiplex artifacts are hard to occur when the ultrasonic image data has a small display depth, or when the transmission pulses have a large PRF.

Therefore, the controller 16 according to a fifth embodiment switches the order of a plurality of ultrasonic transmissions/receptions executed by the transmitter/receiver 11 on a scanning line, in accordance with the display depth or the pulse repetition frequency. Specifically, the controller 16 switches the order of a plurality of ultrasonic transmissions/receptions executed by the transmitter/receiver 11 on a scanning line in an ultrasonic transmission/reception set, in accordance with the display depth or the pulse repetition frequency. More specifically, the controller 16 switches the order of the ultrasonic transmissions/receptions such that a plurality of transmissions corresponding to the reception signals of one set combined by the combining unit 121a are adjacent, in accordance with the display depth or the pulse repetition frequency. In other words, the controller 16 according to the fifth embodiment switches the scan sequence changed based on the restriction on the transmission order to the scan sequence before change, in accordance with the display depth or the pulse repetition frequency.

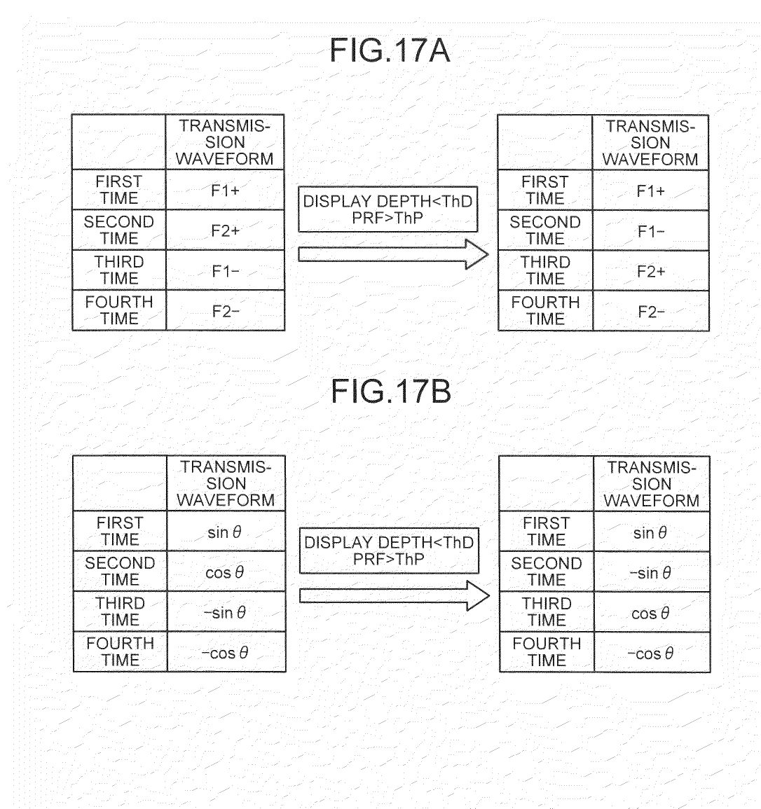

For example, in the transmission order (hereinafter referred to as the “first transmission order”) explained in the first to the fourth embodiments, the transmitter/receiver 11 causes the ultrasonic probe 1 to execute ultrasonic transmissions in the order of “the first ultrasonic pulse, the second ultrasonic pulse, the third ultrasonic pulse, and the fourth ultrasonic pulse”. In contrast, in a conventional transmission order (hereinafter referred to as the “second transmission order”), the transmitter/receiver 11 causes the ultrasonic probe 1 to execute ultrasonic transmissions in the order of “the first ultrasonic pulse, the third ultrasonic pulse, the second ultrasonic pulse, and the fourth ultrasonic pulse”. The transmitter/receiver 11 according to the fifth embodiment switches the first transmission order to the second transmission order, in accordance with the display depth or the pulse repetition frequency. FIG. 17A and FIG. 17B are diagrams for explaining the fifth embodiment.

In FIG. 17A and FIG. 17B, “ThD” denotes a threshold set for the display depth, and “ThP” is a threshold set for the PRF. The thresholds “ThD” and “ThP” may be set as initial setting in the system in advance, or may be set by the operator.

For example, in the first embodiment, the transmission waveforms of the first to the fourth times are “F1+, F2+, F1−, F2−” under the restriction conditions. In the operation, the controller 16 obtains a value of “display depth” set as the display condition, or a value of “PRF” set as the transmission condition. In the case of “display depth<ThD” or “PRF>ThP”, the controller 16 switches to the conventional scan sequence having the transmission waveforms of the first to the fourth times “F1+, F1−, F2+, F2−”, as illustrated in FIG. 17A.

In addition, for example, in the fourth embodiment, the transmission waveforms of the first to the fourth times are “sin θ, cos θ, −sin θ, −cos θ” under the restriction conditions. However, in the case of “display depth<ThD” or “PRF>ThP”, the controller 16 switches to the conventional scan sequence having the transmission waveforms of the first to the fourth times “sin θ, −sin θ, cos θ, −cos θ”, as illustrated in FIG. 17B.

The controller 16 may perform the above switching control, in accordance with a switching request from the operator who has set the display conditions or the transmission conditions.

As described above, according to the fifth embodiment, when the condition is set under which residual multiplex artifacts are hard to occur, the controller 16 switches to a conventional scan sequence with a short interval between transmissions for obtaining two reception signals to be subjected to addition or subtraction to extract a signal component for imaging. As a result, the fifth embodiment enables reduction in occurrence of motion artifacts. When the condition is changed to the condition under which residual multiplex artifacts easily occur (in the case of display depth≧ThD or PRF≦ThP) while the conventional scan sequence is being executed, the controller 16 may switch to the scan sequence including the transmission order based on the restriction conditions. Specifically, the fifth embodiment may include the case of switching the second transmission order to the first transmission order, in accordance with the display depth or the pulse repetition frequency after change.

In the above embodiments, the method for removing residual multiplex artifacts in THI is also applicable to contrast harmonic imaging (CHI) serving as another example of harmonic imaging.

The signal processing and the image generation explained in the above first to fifth embodiments may be executed by an image processing apparatus that is installed independently of the ultrasonic diagnostic apparatus. The image processing apparatus includes an obtaining unit that obtains reception signals generated by the transmitter/receiver 11 by the scan sequence that is set under the control of the controller 16 on the transmission order from the ultrasonic diagnostic apparatus or a storage medium, for example, and a processor that has functions equivalent to those of the signal processor 12 and the image generator 13. The image processing apparatus executes the signal processing and the image generation explained in the above first to fifth embodiments with these processors.

The constituent elements of the apparatuses illustrated in the above first to fifth embodiments are functional conceptual elements, and not always necessary to be physically configured as illustrated. Specifically, the specific form of distribution and integration of each of the apparatuses is not limited to those illustrated, but may be constructed by distributing or integrating the whole or part thereof functionally or physically in a desired unit, according to various loads or state of use. In addition, the whole or part of each processing function executed in each apparatus may be achieved as a CPU or a computer program that is analyzed and executed by the CPU, or hardware using a wired logic.

In addition, the control method explained in the above first to fifth embodiments may be achieved by executing a prepared control program in a computer such as a personal computer and a work station. The control program can be distributed via a network such as the Internet. The control program can be stored in a computer-readable non-transitory storage medium such as a hard disk, a flexible disk (FD), a CD-ROM, an MO, and a DVD, and executed by being read out of the non-transitory storage medium by a computer.

As explained above, the first to the fifth embodiments can remove residual multiplex artifacts.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising:
a transmitter/receiver that executes ultrasonic transmission/reception sets a plurality of times on an identical scanning line by changing transmission conditions, and that generates a plurality of sets of reception signals, the ultrasonic transmission/reception sets including a plurality of ultrasonic transmissions/receptions on the identical scanning line serving as a unit;
a signal processor that combines the plurality of the sets of the reception signals in each of the plurality of the sets, and that generates a plurality of composite signals corresponding to each of the plurality of the sets;
an image generator that generates ultrasonic image data using the plurality of the composite signals; and
a controller that controls an order of ultrasonic transmissions/receptions executed by the transmitter/receiver such that previous transmissions of respective transmissions corresponding to the plurality of the sets of the reception signals in one set combined by the signal processor have an identical transmission condition but different phase polarities.

2. The ultrasonic diagnostic apparatus according to claim 1, wherein the transmission conditions the transmitter/receiver changes at each of the plurality of the sets are at least one of a transmission focus position, a transmission frequency, and a transmission waveform.

3. The ultrasonic diagnostic apparatus according to claim 2, wherein the transmitter/receiver executes the ultrasonic transmission/reception sets a plurality of times on the scanning line at different transmission focus positions, and generates the plurality of the sets of reception signals, the ultrasonic transmission/reception sets including two ultrasonic transmissions/receptions serving as the unit and being executed twice on the identical scanning line with inverted phase polarities,
the signal processor adds two reception signals in each of the plurality of the sets, and generates the plurality of the composite signals on the scanning line; and
the controller controls the transmitter/receiver such that previous transmissions of two respective transmissions corresponding to the two reception signals in one set added by the signal processor have an identical transmission focus position.

4. The ultrasonic diagnostic apparatus according to claim 2, wherein the transmitter/receiver executes the ultrasonic transmission/reception sets a plurality of times on the scanning line with different transmission frequencies, and generates the plurality of the sets of reception signals, the ultrasonic transmission/reception sets including two ultrasonic transmissions/receptions serving as the unit and being executed twice on the identical scanning line with inverted phase polarities,
the signal processor adds two reception signals in each of the plurality of the sets, and generates the plurality of the composite signals on the scanning line;
the image generator generates an ultrasonic image data group using each of the sets of composite signals, and generates image data obtained by combining the ultrasonic image data group as the ultrasonic image data; and
the controller controls the transmitter/receiver such that previous transmissions of two respective transmissions corresponding to the two reception signals in one set added by the signal processor have an identical transmission frequency.

5. The ultrasonic diagnostic apparatus according to claim 2, wherein the transmitter/receiver executes, on the identical scanning line, a first set of ultrasonic transmission/reception including two ultrasonic transmissions/receptions serving as a unit and being executed with inverted phase polarities of an ultrasonic pulse, and a second set of ultrasonic transmission/reception including two ultrasonic transmissions/receptions serving as a unit and being executed with inverted phase polarities of an ultrasonic pulse having a transmission waveform different from a transmission waveform of the ultrasonic pulse of the first set, and generates the two sets of reception signals,
the signal processor generates, on the scanning line, two composite signals including a composite signal obtained by adding two reception signals obtained by the first set of ultrasonic transmission/reception, and a composite signal obtained by adding two reception signals obtained by the second set of ultrasonic transmission/reception, and performs subtraction processing on the two composite signals, and generates a composite signal on the scanning line;
the image generator generates the ultrasonic image data using the composite signal; and
the controller controls the transmitter/receiver such that previous transmissions of two respective transmissions corresponding to the two reception signals in one set added by the signal processor have an identical transmission waveform.

6. The ultrasonic diagnostic apparatus according to claim 2, wherein the transmitter/receiver executes the ultrasonic transmission/reception sets a plurality of times on the scanning line at different transmission focus positions, and generates the plurality of the sets of reception signals, the ultrasonic transmission/reception sets including two ultrasonic transmissions/receptions serving as a unit and executed twice on the identical scanning line with ultrasonic pulses of an identical phase polarity,
the signal processor performs subtraction processing on two reception signals in each of the plurality of the sets, and generates the plurality of composite signals on the scanning line; and
the controller controls the transmitter/receiver such that previous transmissions of two respective transmissions corresponding to the two reception signals in one set subjected to the subtraction processing performed by the signal processor have an identical transmission focus position.

7. The ultrasonic diagnostic apparatus according to claim 1, wherein the controller switches the order of the ultrasonic transmissions/receptions such that a plurality of transmissions corresponding to the reception signals of one set combined by the signal processor are adjacent, in accordance with a display depth or a pulse repetition frequency.

8. An ultrasonic diagnostic apparatus comprising:
a transmitter/receiver
that causes an ultrasonic probe to transmit a first ultrasonic pulse based on a first transmission condition relating to a certain scanning line,
that causes the ultrasonic probe to transmit, subsequent to the first ultrasonic pulse, a second ultrasonic pulse based on a second transmission condition relating to the certain scanning line and being different from the first transmission condition,
that causes the ultrasonic probe to transmit, after the second ultrasonic pulse, a third ultrasonic pulse based on a third transmission condition relating to the certain scanning line and including a phase polarity different from that under the first transmission condition,
that causes the ultrasonic probe to transmit, subsequent to the third ultrasonic pulse, a fourth ultrasonic pulse based on a fourth transmission condition relating to the certain scanning line and including a phase polarity different from that under the second transmission condition, and
that generates a first reception signal based on a reflected wave received by the ultrasonic probe as a result of transmission of the first ultrasonic pulse, a second reception signal based on a reflected wave received by the ultrasonic probe as a result of transmission of the second ultrasonic pulse, a third reception signal based on a reflected wave received by the ultrasonic probe as a result of transmission of the third ultrasonic pulse, and a fourth reception signal based on a reflected wave received by the ultrasonic probe as a result of transmission of the fourth ultrasonic pulse;
a signal processor that generates a first composite signal by combining the first reception signal with the third reception signal, and that generates a second composite signal by combining the second reception signal with the fourth reception signal; and
an image generator that generates image data based on the first composite signal and the second composite signal.

9. The ultrasonic diagnostic apparatus according to claim 8, wherein the transmitter/receiver changes at least one of transmission conditions including a transmission focus position, a transmission frequency, and a transmission waveform between the first transmission condition and the third transmission condition and between the second transmission condition and the fourth transmission condition.

10. The ultrasonic diagnostic apparatus according to claim 9, wherein the transmitter/receiver inverts the phase polarity of the first ultrasonic pulse to obtain the phase polarity of the third ultrasonic pulse, with transmission focus positions of the first ultrasonic pulse and the third ultrasonic pulse serving as a first position, and inverts the phase polarity of the second ultrasonic pulse to obtain the phase polarity of the fourth ultrasonic pulse, with transmission focus positions of the second ultrasonic pulse and the fourth ultrasonic pulse serving as a second position different from the first position, and
the signal processor generates the first composite signal by adding the first reception signal and the third reception signal, and generates the second composite signal by adding the second reception signal and the fourth reception signal.

11. The ultrasonic diagnostic apparatus according to claim 9, wherein the transmitter/receiver inverts the phase polarity of the first ultrasonic pulse to obtain the phase polarity of the third ultrasonic pulse, with transmission frequencies of the first ultrasonic pulse and the third ultrasonic pulse serving as a first frequency, and inverts the phase polarity of the second ultrasonic pulse to obtain the phase polarity of the fourth ultrasonic pulse, with transmission frequencies of the second ultrasonic pulse and the fourth ultrasonic pulse serving as a second frequency different from the first frequency, and
the signal processor generates the first composite signal by adding the first reception signal and the third reception signal, and generates the second composite signal by adding the second reception signal and the fourth reception signal.

12. The ultrasonic diagnostic apparatus according to claim 9, wherein
the transmitter/receiver inverts the phase polarity of the first ultrasonic pulse to obtain the phase polarity of the third ultrasonic pulse, with transmission waveforms of the first ultrasonic pulse and the third ultrasonic pulse serving as a first waveform, and inverts the phase polarity of the second ultrasonic pulse to obtain the phase polarity of the fourth ultrasonic pulse, with transmission waveforms of the second ultrasonic pulse and the fourth ultrasonic pulse serving as a second waveform different from the first waveform,
the signal processor generates the first composite signal by adding the first reception signal to the third reception signal, generates the second composite signal by adding the second reception signal to the fourth reception signal, and generates a composite signal by performing subtraction processing on the first composite signal and the second composite signal, and
the image generator generates the image data based on the composite signal.

13. The ultrasonic diagnostic apparatus according to claim 9, wherein the transmitter/receiver inverts the phase polarity of the first ultrasonic pulse to obtain the phase polarity of the third ultrasonic pulse, with transmission focus positions of the first ultrasonic pulse and the third ultrasonic pulse serving as a first position, and inverts the phase polarity of the second ultrasonic pulse to obtain the phase polarity of the fourth ultrasonic pulse, with transmission focus positions of the second ultrasonic pulse and the fourth ultrasonic pulse serving as a second position different from the first position, and
the signal processor generates the first composite signal by performing subtraction processing on the first reception signal and the third reception signal, and generates the second composite signal by performing subtraction processing on the second reception signal and the fourth reception signal.

14. The ultrasonic diagnostic apparatus according to claim 8, wherein the transmitter/receiver switches a first transmission order to a second transmission order, in accordance with a display depth or a pulse repetition frequency, the first transmission order causing the ultrasonic probe to execute ultrasonic transmission in an order of the first ultrasonic pulse, the second ultrasonic pulse, the third ultrasonic pulse, and the fourth ultrasonic pulse, and the second transmission order causing the ultrasonic probe to execute ultrasonic transmission in an order of the first ultrasonic pulse, the third ultrasonic pulse, the second ultrasonic pulse, and the fourth ultrasonic pulse.

15. An ultrasonic diagnostic apparatus comprising:
a transmitter/receiver that executes ultrasonic transmission/reception sets a plurality of times on an identical scanning line by changing transmission conditions, and that generates a plurality of sets of reception signals, the ultrasonic transmission/reception sets including a plurality of ultrasonic transmissions/receptions on the identical scanning line serving as a unit;
a signal processor that combines the plurality of the sets of the reception signals in each of the plurality of the sets, and that generates a plurality of composite signals corresponding to each of the plurality of the sets;
an image generator that generates ultrasonic image data using the plurality of the composite signals; and
a controller that switches an order of the plurality of the ultrasonic transmissions/receptions executed by the transmitter/receiver on the scanning line, in accordance with a display depth or a pulse repetition frequency.

16. A control method including:
executing, by a transmitter/receiver, ultrasonic transmission/reception sets a plurality of times on an identical scanning line by changing transmission conditions, and generating a plurality of sets of reception signals, the ultrasonic transmission/reception sets including a plurality of ultrasonic transmissions/receptions on the identical scanning line serving as a unit;
combining, by a signal processor, the plurality of the sets of the reception signals in each of the plurality of the sets, and generating a plurality of composite signals corresponding to each of the plurality of the sets;
generating, by an image generator, ultrasonic image data using the plurality of the composite signals; and
controlling, by a controller, an order of ultrasonic transmissions/receptions executed by the transmitter/receiver such that previous transmissions of respective transmissions corresponding to the plurality of the sets of the reception signals in one set combined by the signal processor have an identical transmission condition but different phase polarities.
